(19) United States
(12) Patent Application Publication
(10) Pub. No.: US 2013/0302896 A1
(43) Pub. Date: Nov. 14, 2013

(54) SOY-PROTEIN CONTAINING POROUS MATERIALS

(71) Applicant: Northwestern University, Evanston, IL (US)

(72) Inventors: Ramille N. Shah, Newark, NJ (US); Karpagavalli Sundaresan, Chicago, IL (US); Karen B. Chien, Chicago, IL (US)

(21) Appl. No.: 13/798,609

(22) Filed: Mar. 13, 2013

Related U.S. Application Data
(60) Provisional application No. 61/614,648, filed on Mar. 23, 2012.

(51) Int. Cl. A61L 27/60; C12N 5/0775; A61L 27/22

(52) U.S. Cl. CPC A61L 27/60; A61L 27/22; C12N 5/0675
USPC 435/399; 623/23.72; 530/378; 264/6

(57) ABSTRACT

Porous soy protein-based materials are provided. Also provided are tissue growth scaffolds comprising the porous soy protein-based materials. Methods for forming the porous soy protein-based materials and methods for growing tissue on the tissue growth scaffolds are also provided. The porous soy protein-based materials comprise a plurality of soy protein chains that are crosslinked in a three-dimensional structure that provides a high degree of porosity. In order to achieve highly porous structures, the soy proteins, which are globular in nature, can be partially denatured in order to facilitate crosslinking between, and entanglement of, the soy protein chains.

### CROSS-REFERENCE TO RELATED APPLICATIONS

This application claims priority from U.S. provisional patent application Ser. No. 61/614,648, filed on Mar. 23, 2012, which is incorporated herein by reference.

### BACKGROUND

Tissue engineering involves the fabrication of constructs which aid in the repair and regeneration of damaged tissue, providing proper structure, function and integration with the host tissue. One frontier of tissue engineering lies in using a biomaterial scaffold to deliver cell-based therapy. Porous scaffolds provide three-dimensional microenvironments, which can mimic the extracellular matrix and can allow for cell infiltration and space for matrix deposition by cells to form new tissue. An ideal scaffold material should stimulate the formation of tissue which is structurally and functionally robust, while being safe and cost-efficient to obtain, process and manufacture. The use of natural proteins to form biomaterials is an attractive therapy because of the ability of the natural material to control stem cell adhesion and growth through inherent binding sites.

Human mesenchymal stem cells (hMSC) seeded on collagen and silk protein scaffolds have been shown to proliferate and differentiate into osteoblasts and chondrocytes that were fully functional, biocompatible and able to form tissues resembling native tissue structure and function. In addition, soy curd containing protein and other soybean components have been shown to decrease the level of proinflammatory cytokine production of mononuclear cells from human peripheral blood and to promote osteoblast proliferation. (Santin M, Morris C, Standen G, Nicolais L, Ambrosio L. A new class of bioactive and biodegradable soybean-based bone fillers. Biomacromolecules 2007; 8:2706-11.)

### SUMMARY

Porous soy protein-containing materials comprising a plurality of partially denatured soy protein chains are provided. In the materials, at least some of the soy protein chains are entangled and crosslinked to other soy protein chains. The soy protein chains can be crosslinked with enzymatic crosslinkers or with non-enzymatic chemical crosslinkers. The materials have high porosities and pore sizes suitable for promoting cell growth and proliferation within the pores.

Tissue growth scaffolds are also provided. These scaffolds comprise a porous soy protein-containing material comprising a plurality of partially denatured soy protein chains, wherein at least some of the soy protein chains are entangled and crosslinked to other soy protein chains; and tissue-forming cells, or cells that are precursors to tissue-forming cells, integrated within the pores of the porous soy protein-containing material.

Also provided are methods of growing tissue on a tissue growth scaffold, the tissue growth scaffold comprising a porous soy protein-containing material comprising a plurality of partially denatured soy protein chains, wherein at least some of the soy protein chains are entangled and crosslinked to other soy protein chains. The methods comprise incorporating tissue-forming cells, or cells that are precursors to tissue-forming cells, into the tissue growth scaffold; and culturing the seeded cells in a cell growth culture medium.

Also provided are methods of forming porous soy protein-containing material comprising a plurality of partially denatured soy protein chains, wherein at least some of the soy protein chains are entangled and crosslinked to other soy protein chains. In one embodiment, the method comprises reacting a crosslinker with partially denatured soy protein chains in a slurry comprising the soy protein chains, the crosslinker and a solvent under conditions that provide covalent linkages between at least some of the soy protein chains; and lyophilizing the slurry to provide the porous soy protein-containing material. In another embodiment, the method comprises electrospinning a first solution comprising partially denatured soy protein chains and organic fiber-forming molecules under conditions that provide a mat of nanofibers, wherein the nanofibers comprise the soy protein chains and the organic fiber-forming molecules; and crosslinking the nanofibers in the mat by exposing them to a second solution comprising a crosslinker under conditions that provide covalent linkages between at least some of the soy protein chains of at least some nanofibers with soy protein chains of other nanofibers, thereby providing the porous soy protein-containing material.

Other principal features and advantages of the invention will become apparent to those skilled in the art upon review of the following drawings, the detailed description, and the appended claims.

### BRIEF DESCRIPTION OF THE DRAWINGS

Illustrative embodiments, which are not intended to be limiting, of the invention will hereafter be described with reference to the accompanying drawings.

FIG. 1 depicts the reaction between glutamine and lysine side chains facilitated by transglutaminase.

FIG. 2 shows (A) a protocol used to freeze soy protein-containing slurries. Slurries were frozen for at least 5 hours to ensure complete solidification before drying and (B) the types of scaffolds (i.e., porous soy protein-containing materials) fabricated.

FIG. 3 shows scanning electron microscopy images of scaffolds. (A) Dry and (B) hydrated 5% soy protein isolate (SPI) one unit enzyme-treated representative scaffolds. Controls appear similar to (A, B). (C) Dry and (D) hydrated 3% SPI twenty unit enzyme-treated scaffolds, which are representative of all twenty unit enzyme-treated scaffolds.

FIG. 4 shows (A) the pore size distribution of representative 5% SPI scaffold groups according to an exemplary embodiment and (B) the total percent of occupied pore space within scaffolds. Data shown are mean±S.E.M. with n=3. (*: p<0.05)

FIG. 5 shows the moisture content of scaffolds. HT control (heat treatment only) and MD 1U control (one unit maltodextrin only) are scaffold groups with slurry heat treatment and maltodextrin only controls respectively. TG 1U (transglutaminase one unit) and TG 20U (transglutaminase twenty units) are scaffold groups with transglutaminase/maltodextrin additives with one unit and twenty units added respectively. Data shown are mean±S.E.M with n=5. (*: p<0.05; **: p<0.01; ***: p<0.001)

FIG. 6 shows the mechanical properties of scaffolds. (A) Compressive modulus of dry scaffolds. (B) Compressive modulus of hydrated scaffolds. Data shown are mean±S.E.M. with n=4-5. (*: p<0.05; **: p<0.01; ***: p<0.001)

FIG. 7 shows DNA quantification per scaffold over various time points. (A) HT control scaffolds. (B) MD control scaffolds. (C) TG 1U scaffolds. (D) TG 20U scaffolds. (*: p<0.05; **: p<0.01)

FIG. 8 shows the relative weight fraction for 3% SPI scaffolds over time. Cell integration is observed for scaffolds where the relative fraction has dropped below approximately 0.9, while a cell sheet forms for relative weight fractions that are greater than approximately 0.9. (A) 3% SPI scaffolds. (B) 5% SPI scaffolds.

FIG. 9 shows the influence of applied voltage on electrospun fiber morphology of 7% SPI/5% 100 kDa PEO (poly(ethylene oxide)) at (a) 15 kV (b) 20 kV, (c) 27 kV and (d) variation in average fiber diameter.

FIG. 10 shows the change in electrospun fiber morphology with SPI concentration of (a) 5% SPI/5% 100 kDa PEO, (b) 7% SPI/5% 100 kDa PEO, (c) 10% SPI/5% 100 kDa PEO, (d) 12% SPI/5% 100 kDa PEO, (e) variation in average fiber diameter, and (f) surface tension and viscosity.

FIG. 11 shows the change in electrospun fiber morphology with PEO concentration of (a) 5% 100 kDa PEO/7% SPI, (b) 10% 100 kDa PEO/7% SPI, (c) 15% 100 kDa PEO/7% SPI, (d) 20% 100 kDa PEO/7% SPI (e) variation in average fiber diameter, and (f) surface tension and viscosity.

FIG. 12 shows the influence of PEO molecular weight on electrospun fiber morphology at (a) 3% 100 kDa PEO/7% SPI, (b) 3% 1MDa PEO/7% SPI, (c) 3% 8MDa PEO/7% SPI, (d) higher magnification of 3% 1MDa PEO/7% SPI electrospun fiber which is used for tensile tests and cell culture studies, (e) variation in average fiber diameter, and (f) surface tension and viscosity.

FIG. 13 shows the electrospun fiber morphology after EDC/NHS (ethyl-3-(dimethylaminopropyl)-carbodiimide/N-hydroxysuccinimide) crosslinking of (a) 7% SPI/3% 1MDa PEO, (b) 12% SPI/3% 1MDa PEO, (c) change in average fiber diameter, and (d) porosity before and after EDC/NHS crosslinking.

FIG. 14 shows PicoGreen dsDNA quantization of electrospun scaffolds 7% SPI/3% PEO and 12% SPI/3% PEO after 1 and 7 days of culture.

FIG. 15 shows human mesenchymal stem cells (hMSCs) attachment and spreading on electrospun scaffolds (a) 7% SPI/3% PEO and (b) 12% SPI/3% PEO after (i) 1 and (ii) 7 days of culture.

### DETAILED DESCRIPTION

Porous soy protein-based materials are provided. Also provided are tissue growth scaffolds comprising the porous soy protein-based materials. Methods for forming the porous soy protein-based materials and methods for growing tissue on the tissue growth scaffolds are also provided.

The porous soy protein-based material comprises a plurality of soy protein chains that are crosslinked in a three-dimensional structure that provides a high degree of porosity. The soy proteins, which are globular in nature, can be partially denatured in order to facilitate crosslinking between, and entanglement of, the soy protein chains. In some embodiments, the material comprises substantially no other plant-based protein or polymer besides soy protein. In some embodiments, the material does not comprise any other plant-based protein or polymer besides soy protein. In some embodiments, the material comprises substantially no cellulose or chitosan. In some embodiments, the material does not comprise cellulose or chitosan.

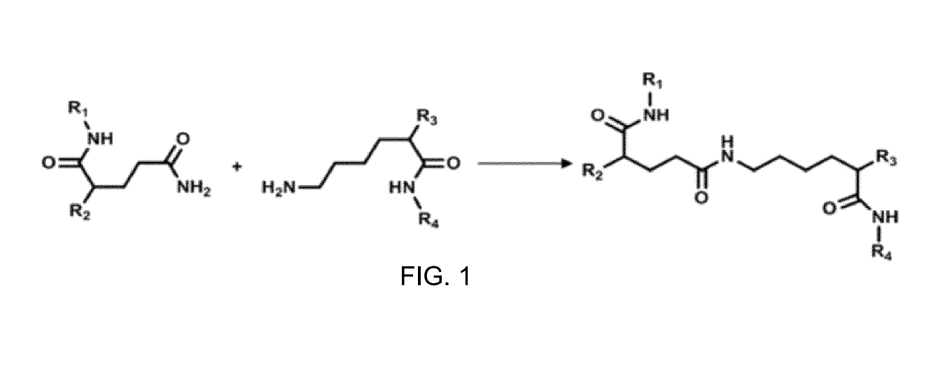

The soy protein chains can be crosslinked by molecules capable of reacting with functional groups on the soy protein chains, such as —NH₂, —OH and —SH groups, to form covalent linkages between the chains, e.g., between two different chains. In some embodiments, the crosslinkers are enzymatic crosslinkers. In some embodiments, the crosslinkers are non-enzymatic chemical molecules. Transglutaminase is one example of an enzymatic crosslinker. Organic molecules, such as carbodiimides (e.g., ethyl-3-(dimethylaminopropyl)-carbodiimide, EDC) and N-hydroxysuccinimide (NHS) are examples of non-enzymatic, chemical crosslinkers. As shown in FIG. 1, transglutaminase reacts with soy protein chains to facilitate the reaction of the γ-carboxamide group of a glutamine side chain with the e-amino group of a lysine side chain to form a ε(γ-glutamyl) lysine linkage. Thus, in some embodiments, at least some of the soy protein chains are crosslinked to other soy protein chains in the material via one or more ε(γ-glutamyl) lysine linkages. The non-enzymatic chemical crosslinker ethyl-3-(dimethylaminopropyl)-carbodiimide) reacts with carboxylic acid groups to form an active o-acylisourea intermediate, which is subsequently displaced by nucleophilic attack from primary amino groups to form an amide linkage. The presence of N-hydroxysuccinimide improves the efficiency of this reaction by stabilizing the o-acylisourea intermediate, which is also subsequently displaced by primary amino groups to form an amide linkage. Thus, in some embodiments, at least some of the soy protein chains are crosslinked to other soy protein chains in the material via one or more amide linkages.

Other enzymatic crosslinkers and non-enzymatic crosslinkers may be used. Other examples of non-enzymatic crosslinkers include genipin, glutaraldehyde, glyoxal, tannic acid, and formaldehyde. In some embodiments, at least some of the soy protein chains are crosslinked to other soy protein chains in the material via the covalent linkages afforded by these crosslinkers. However, in some embodiments, the crosslinker is not genipin, glutaraldehyde, glyoxal, tannic acid, or formaldehyde.

Other enzymatic crosslinkers include oxidative enzymes, polyphenoloxidases (PPOs), such as tyrosinase and laccases (Buchert et al., Annu Rev Food Sci Technol Vol. 1: 113-138 (2010), which is hereby incorporated by reference in its entirety). Tyrosinase acts by catalyzing ortho-hydroxylation of monophenols and oxidation of diphenols; this enzyme is capable of reacting tyrosine with lysyl, tyrosyl, and cysteinyl residues between proteins. Laccases catalyze oxidation in phenols using single electron removal. Specific reactions between tyrosine and other free aromatic and amino groups from other proteins such as cysteine and tryptophan occur when free radicals are generated from oxidation.

Soy protein chains crosslinked with crosslinker molecules may be distinguished from soy protein chains that may become crosslinked even in the absence of such molecules, e.g., through the formation of disulfide linkages. In some embodiments, substantially no soy protein chains are crosslinked to other soy protein chains in the material via disulfide linkages. However, in some embodiments, some soy protein chains are crosslinked to other soy protein chains in the material via disulfide linkages, e.g., in additional to linkages formed via the disclosed crosslinkers.

Porous soy protein-based materials having various degrees of crosslinking, i.e., various relative crosslinking densities, may be used. In some embodiments, the relative crosslinking density of the material is at least about 0.1. This includes embodiments in which the relative crosslinking density is at least about 0.2, at least about 0.3, at least about 0.4 or at least about 0.6. The relative crosslinking density may be adjusted, e.g., by adjusting the amount of crosslinker used to form the material as well as the reaction conditions under which the crosslinking reaction occurs. Exemplary suitable amounts and reaction conditions are described below. Relative crosslinking densities may be evaluated using the method described in Vickers S M, Squitieri L S, Spector M. “Effects of Cross-linking Type II Collagen-GAG Scaffolds on Chondrogenesis In Vitro: Dynamic Pore Reduction Promotes Cartilage Formation.” Tissue Eng. 2006; 12(5):1345-55.

The porous soy protein-based materials may be used as growth scaffolds for tissues. An embodiment of a tissue growth scaffold comprises the porous soy protein-containing material of the types described herein and a plurality of tissue forming cells, or cells that are precursors to tissue-forming cells, integrated into the material, such that the cells are located and attached within the pores of the material.

The tissue growth scaffolds can be used to grow a tissue by incorporating tissue-forming biological cells, or biological cells that are precursors to tissue-forming cells into the tissue growth scaffold (e.g., by seeding the cells), and culturing the seeded cells in a cell growth culture medium. Human mesenchymal stem cells (hMSC), hematopoetic stem cells, embryonic stem cells, and induced pluripotent stem cells are examples of precursors to tissue-forming cells. Examples of tissue-forming cells include osteoblasts, chondrocytes, fibroblasts, endothelial cells and myocytes. Exemplary suitable techniques and conditions for seeding and culturing cells are described in the Examples, below. The methods of growing a tissue may be carried out in vitro, ex vivo or in vivo.

Soy protein is based on amino acids of aspartic acid (aspargine) and glutamic acid (glutamine), nonpolar amino acids (glycine, alanine, valine, and leucine), basic amino acids (lysine and arginine), and small amounts of cysteine. The use of soy proteins is advantageous due to its ease of processing during structural fabrication. In addition, the structural, mechanical, and biological properties of soy protein biomaterials can be tuned through thermal and chemical treatments during processing. This is due, at least in part, to the presence of reactive groups, such as —NH₂, —OH and —SH, which make soy proteins suitable for chemical, physical, thermal and enzymatic modifications and make available versatile routes to tailor the protein properties for the diverse requirements of specific biomedical applications. In addition, soy proteins do not have an animal origin, are non-cytotoxic and biodegradable, are economically competitive, and have good water resistance and storage stability as compared to other biodegradable polymers and natural proteins available for biomedical applications. The combination of these properties with a similarity to tissue constituents and reduced susceptibility to thermal degradation, which allows easy processing into three-dimensional (3D) shapes, make soy proteins a useful material for tissue engineering scaffolds.

The present soy protein-based porous materials can take a variety of forms. By way of illustration only, in some embodiments, the materials are thin films. Such films can be fabricated by casting a slurry comprising the soy protein in a solvent, such as a glycerol water mixture, into a mold and drying the slurry.

Alternatively, the soy protein-based porous materials can be porous networks formed from lyophilized slurries of soy proteins. In some embodiments, a method for forming such a material comprises reacting a crosslinker with soy protein in a slurry comprising the soy protein, the crosslinker and a solvent (e.g., water) under conditions that provide covalent linkages between soy proteins, followed by lyophilizing the slurry to a low moisture content (e.g., a moisture content of no greater than about 10 wt. %). In another embodiment, a method comprises lyophilizing a slurry comprising soy protein and a solvent, followed by reacting a crosslinker with the soy protein in the lyophilized slurry under conditions that provide covalent linkages between soy proteins. Slurries comprising soy proteins can be treated to partially denature the soy proteins prior to crosslinking or prior to lyophilization (e.g., by application of heat at a temperature that avoids decomposition of the soy protein). Exemplary suitable conditions for crosslinking reactions, denaturing soy protein and lyophilization are described below and/or in Example 1.

Various amounts of soy protein may be used in the slurries. For tissue engineering applications, it is desirable for the soy proteins of the disclosed porous materials to degrade in the cell growth medium at a rate that is sufficiently slow to allow cells to proliferate and to integrate into the pores of the material. In some embodiments, the amount of soy protein is that which provides a material containing the soy protein having a desired degradation time (e.g., greater than about seven days or at least about fourteen days). In some embodiments, the amount of soy protein is that which provides a material capable of supporting the proliferation of cells (e.g., human mesenchymal stem cells) in the material for a desired period of time (e.g., at least about seven days or at least about fourteen days). In some embodiments, the amount of soy protein is that which provides a material capable of becoming integrated with cells in the pores of the material (e.g., as measured at a particular time point, e.g., at about seven days or at about fourteen days). In some embodiments, the amount of soy protein in the slurry is in the range from about 1 weight % to about 10 weight %. By “weight %” it is meant the percent by weight of the soy protein compared to the total weight of the slurry (e.g., a slurry of soy protein and a solvent). This includes embodiments in which the amount is in the range of from about 1 weight % to about 5 weight % or from about 3 weight % to about 5 weight %.

Various types (e.g., as described above) and amounts of crosslinker may be used in the slurries. In some embodiments, the crosslinker is an enzymatic crosslinker. In some embodiments, the amount of enzymatic crosslinker is that which provides a material having a desired degradation time (e.g., greater than about seven days or at least about fourteen days). In some embodiments, the amount of enzymatic crosslinker is that which provides a material capable of supporting the proliferation of cells (e.g., human mesenchymal stem cells) in the material for a desired period of time (e.g., about seven days or at least about fourteen days). In some embodiments, the amount of enzymatic crosslinker is that which provides a material capable of becoming integrated with cells in the pores of the material (e.g., as measured at a particular time point, e.g., at about seven days or at about fourteen days). For enzymatic crosslinkers, the amount of crosslinker may be quantified as the units of enzyme activity per gram of soy protein. In some embodiments, the amount of enzymatic crosslinker is in the range of from about 0.1 units of enzyme activity per gram of soy protein to about 25 units of enzyme activity per gram of soy protein. This includes embodiments in which the amount is in the range of from about 1 unit to about 20 units, from about 1 unit to about 10 units, from about 1 unit to about 5 units, from about 1 unit to about 3 units, from about 0.1 units to about 10 units, from about 0.1 units to about 5 units, or from about 0.1 units to about 1 unit.

In some embodiments, the amount of soy protein in the slurry is in the range of from about 1 weight % to about 10 weight % and the amount of enzymatic crosslinker in the slurry is in the range of from about 0.1 units of enzyme activity per gram of soy protein to about 25 units. This includes embodiments in which the amount of soy protein is in the range of from about 3 weight % to about 5 weight % and the amount of enzymatic crosslinker is in the range of from about 1 unit to about 20 units. This further includes embodiments in which the amount of soy protein is about 3 weight % and the amount of enzymatic crosslinker is about 1 unit.

Other additives may be used in the slurries in various amounts. For example, the slurry may comprise a plasticizer to decrease the glass transition temperature of the slurry. Glycerol is an exemplary plasticizer. As another example, the slurry may comprise an oligosaccharide to increase cell metabolism. Maltodextrin is an exemplary oligosaccharide. These additives may be present in the resulting soy protein-based porous materials.

Lyophilization of the slurries may be carried out on commercially available lyophilizers. Lyophilization parameters such as freezing temperature, freezing rate, freezing time, drying temperature, drying pressure and drying time may be adjusted to modify the properties of the soy protein-based porous materials (e.g., porosity, pore size, pore shape, compressive modulus). Exemplary parameters are described in Example 1, below. In some embodiments, the lyophilization step does not comprise use of a unidirectional temperature gradient during the freezing process to form unidirectional pores.

In other exemplary embodiments, the soy protein-based porous materials are nanofibrous materials that take the form of a mat of entangled, non-woven fibers, wherein the fibers comprise the soy protein chains. In such embodiments, the fibers may further comprise one or more fiber-forming molecules, such as organic polymers, that facilitate fiber formation. Exemplary organic polymers include poly(dioxanone) (PDO), poly(ethylene oxide) (PEO), poly(ethylene terephthalate), poly(glyconate), poly(D,L-lactideco-glycolide) (PLGA), poly(L-lactide) (PLLA), poly(D,L-lactide-co-ε-caprolactone) (PLCL), poly(styrene), poly(vinyl alcohol) (PVA), chitosan, and hyaluronic acid (HA). The weight ratio of soy protein to fiber-forming molecules in the fibers may vary as further described below. The fibers are desirably continuous fibers, by which it is meant the fibers have a substantially uniform diameter along their lengths and are substantially free of beads, spindles, or similar large diameter (i.e., relative to the remaining segments of the fibers) masses along their lengths. For example, in some embodiments, the diameter of the fibers deviates by no more than about ±30% along their length. This includes embodiments in which the diameter of the strands deviates by no more than about ±20%, no more than about ±10% or no more than about ±5%. The spaces between the entangled, non-woven fibers form the pores of the materials. As further described below, the diameters of the fibers can be tailored to provide a desired porosity and/or average pore size in the resulting material. In some embodiments, the fibers have an average diameter in the range from about 10 nm to about 500 nm. This includes embodiments in which the fibers have an average diameter in the range from about 50 nm to about 300 nm, from about 20 nm to about 200 nm, or from about 90 nm to about 200 nm. The term “average diameter” may refer to the average value of the diameters of population of fibers within the material.

Soy protein-based porous materials in the form of a mat of entangled, non-woven fibers can be fabricated using the technique of electrospinning. In some embodiments, a method of forming such a material comprises electrospinning a first solution (e.g., an aqueous solution) comprising soy proteins and fiber-forming molecules to form a non-woven fiber mat, wherein the fibers comprise the soy protein chains. The method may further comprise crosslinking the fibers in the mat by exposing them to a second solution (e.g., a non-aqueous solution) comprising crosslinkers under conditions that provide covalent linkages between the fibers, e.g., between soy proteins of different fibers. The method may further comprise treating the first solution to partially denature the soy proteins prior to electrospinning (e.g., by application of heat at a temperature that avoids decomposition of the soy protein and/or by adjusting the pH, e.g., with NaOH, to a pH that avoids decomposition of the soy protein). Exemplary suitable conditions for crosslinking reactions, denaturing soy protein and electrospinning are described below and/or in Example 2.

Various amounts of soy protein, amounts of fiber-forming molecules and weight ratios of soy protein to fiber-forming molecules in the first solution may be used. In some embodiments, the amounts and ratios are sufficient to provide continuous fibers having a desired average diameter and/or a material having a desired pore size and/or a desired porosity. In some embodiments, the amount of soy protein in the first solution is in the range from about 1 weight % to about 20 weight %. By “weight %” it is meant the percent by weight of the soy protein compared to the total weight of the first solution. This includes embodiments in which the amount of soy protein is in the range of from about 1 weight % to about 15 weight %, or from about 5 weight % to about 15 weight %. In some embodiments, the amount of fiber-forming molecules in the first solution is in the range from about 1 weight % to about 30 weight %. This includes embodiments in which the amount of fiber-forming molecules is in the range of from about 1 weight % to about 25 weight %, or from about 5 weight % to about 20 weight %. In some embodiments, the weight ratio of soy protein to fiber-forming molecules in the first solution is in the range of from about 25:1 to about 1:25. This includes embodiments in which the weight ratio is in the range from about 20:1 to about 1:20, from about 15:1 to about 1:15, from about 10:1 to about 1:10, from about 5:1 to about 1:5, from about 4:1 to about 1:4, from about 3:1 to about 1:3, from about 2:1 to about 1:2, and about 1:1. This further includes embodiments in which the weight ratio is in the range from about 4:1 to about 1:1 and about 3:1 to about 1:1.

Fiber-forming molecules having various molecular weights may be used. In some embodiments, the molecular weight of the fiber-forming molecules is sufficient to provide continuous fibers having a desired average diameter and/or a material having a desired pore size and/or a desired porosity. In some embodiments, the molecular weight of the fiber-forming molecules is in the range of from about 10 kDa to about 20 MDa. This includes embodiments in which the molecular weight is in the range of from about 100 kDa to about 10 MDa.

In some embodiments, the amount of soy protein, the amount of fiber-forming molecules, the weight ratios of soy protein to fiber-forming molecules and/or the molecular weight of the fiber-forming molecules in the first solution are sufficient to provide a material capable of supporting the proliferation of cells (e.g., human mesenchymal stem cells) in the material for a desired period of time (e.g., at least about seven days). In some embodiments, the amount of soy protein, the amount of fiber-forming molecules, the weight ratios of soy protein to fiber-forming molecules and/or the molecular weight of the fiber-forming molecules in the first solution are sufficient to provide a material capable of becoming integrated with cells in the pores of the material (e.g., as measured at a particular time point, e.g., at about seven days).

In the second solution, a single kind of crosslinker or blends of different crosslinkers may be used. Various amounts of crosslinker, as measured by the molar ratios of crosslinker(s) to functional groups (e.g., COOH), on the soy protein may be used. In some embodiments, the molar ratio is sufficient to provide fibers having a desired average diameter. In some embodiments, the molar ratio is sufficient to provide a material capable of supporting the proliferation of cells (e.g., human mesenchymal stem cells) in the material for a desired period of time (e.g., at least about seven days). In some embodiments, the molar ratio is sufficient to provide a material capable of becoming integrated with cells in the pores of the material (e.g., as measured at a particular time point, e.g., at about seven days).

Commercially available or custom-built apparatuses for carrying out the technique of electrospinning may be used. Electrospinning parameters such as solution flow rate, working distance between needle and collector, and applied voltage may be adjusted, e.g., to provide continuous fibers having a desired average diameter.

A soy protein-based porous material intended for use as a tissue growth template desirably has a high surface area on which to grow tissue, as well as pore sizes sufficient to provide adequate space for cell adhesion, migration, and distribution within the pores to facilitate homogeneous tissue formation throughout the scaffold. Cell integration is preferred over cell sheet formation since cells can grow into a three-dimensional space rather than being limited to confluence in a flat, two-dimensional sheet. Thus, the optimal dimension of the pores and porosity of the material may depend, at least partially, on the nature of the cells to be grown. Generally, it is desirable for the pores to have diameters of at least about 2 times (e.g., about 2-5 times) the diameter of the cells being grown. The pore diameter may refer to a two-dimensional cross-sectional diameter as determined by taking a cross-section through the material and imaging the surface of that section, using, e.g., scanning tunneling microscopy as described in the Examples, below. If multiple cross-sections of a pore are imaged resulting in different diameter values for the pore, the two-dimensional cross-sectional diameter may be taken as the largest of these values. The pores of the material may be characterized by a median or average pore diameter. The terms “median pore diameter” and “average pore diameter” may refer to the median or average value of the pore diameters of a population of pores within the material. By way of illustration only, some embodiments of the materials have a median or average pore diameter of about 200 μm or less. This includes embodiments having a median or average pore diameter of about 150 μm or less, about 100 μm or less, or 50 μm or less. This includes embodiments having a median or average pore diameter in the range of from about 20 μm to about 200 μm, from about 20 μm to about 100 μm, or from about 20 μm to about 60 μm. In other embodiments, the median or average pore diameter is in the range of from about 50 μm to about 1000 μm or from about 100 μm to about 500 μm. The amount of crosslinker may be adjusted to modify the pore diameter.

The pores in the soy protein-based porous material may assume a variety of shapes. Pores may assume spherical and non-spherical (e.g., ellipsoidal or cylindrical) shapes. Pores may assume irregular shapes (e.g., elongated, tortuous channels). The materials may comprise pores of different shapes, e.g., both spherical and irregularly shaped pores. However, in some embodiments, substantially all the pores of the material are irregularly shaped in the form of elongated, tortuous channels. For non-spherically shaped and irregularly shaped pores, the pore diameter may be taken as the largest dimension across the pore. In some embodiments, the orientation of pores throughout the material is substantially random such that the longitudinal axes of the pores are randomly oriented with respect to a plane defined by the outer surface of the material. Both materials having irregularly shaped pores and substantially randomly oriented pores may be distinguished from porous materials formed using lyophilization steps comprising use of a unidirectional temperature gradient during the freezing process to form unidirectional, aligned pores.

The porosity of the soy protein-based porous materials refers to the percentage of void space within the material. By way of illustration, some embodiments of the materials have a porosity of at least about 40 vol. %. This includes embodiments of the materials having a porosity of at least about 50 vol. % and further includes embodiments of the materials having a porosity of at least about 60 vol. %, at least about 70 vol. %, at least about 80 vol. %, or at least about 90 vol. %. Porosity values may be determined using the techniques described in the Examples, below.

A soy protein-based porous material intended for use as a tissue growth scaffold is also desirably sufficiently robust to support the viability and proliferation of cells cultured thereon and, as such, is desirably elastic or viscoelastic. Robustness can be defined by the compressive modulus of the material. The compressive modulus of the material may be determined using the techniques described in Example 1 below. In some embodiments, the materials have a compressive modulus of at least about 30 Pa. This includes embodiments in which the materials have a compressive modulus of at least about 40 Pa, at least about 50 Pa, at least about 60 Pa, or at least about 70 Pa. In some embodiments, the materials have a compressive modulus in the range from about 10 to about 1000 Pa. This includes embodiments in which the compressive modulus is in the range from about 30 to about 100 Pa, from about 20 to about 100 Pa, or from about 30 to 70 Pa. Robustness can also be defined by the Young's modulus of the material. The Young's modulus of the material may be determined using the techniques described in Example 2 below. In some embodiments, the materials have a Young's modulus of at least about 50 kPa. This includes embodiments in which the materials have a Young's modulus of at least about 75 kPa, at least about 100 kPa, or at least about 125 kPa. In some embodiments, the materials have a Young's modulus in the range from about 50 to about 250 kPa. This includes embodiments in which the Young's modulus is in the range from about 100 to about 200 kPa.

The thickness of the soy protein-based porous material is not limited and can depend, at least partially, on the desired application of the material, e.g., on the type and degree of tissue repair and regeneration required. In some embodiments, the thickness of the material is at least 1 mm. This includes embodiments in which the thickness is in the range of from about 1 mm to about 5 mm and from about 1 mm to about 4 mm.

As noted above, for tissue engineering applications, it is desirable for the soy proteins of the porous soy protein-based materials to degrade in the cell growth medium at a rate that is sufficiently slow to allow cells to proliferate and to integrate into the pores of the material. Thus, in some embodiments, the soy protein-based porous material exhibits a degradation time in cell growth medium of at least about seven days. This includes embodiments in which the material exhibits a degradation time in cell growth medium of at least about fourteen days. In some embodiments, the soy protein-based porous material is capable of supporting the proliferation of cells in a cell growth medium for at least about seven days. This includes embodiments in which the material is capable of supporting the proliferation of cells in a cell growth medium for at least about fourteen days. In some embodiments, the soy protein-based porous material is capable of becoming integrated with cells in the pores of the material over a period of at least about seven days. This includes embodiments in which the material is capable of becoming integrated with cells in the pores of the material over a period of at least about fourteen days.

Certain portions of the description above refer to degradation time in a cell growth medium, cell proliferation in a cell growth medium and cell integration in a cell growth medium. Regarding “degradation time,” this phrase may be defined as the time it takes for the soy protein-based porous material to completely degrade in the cell growth medium. The phrase “completely degrade” may refer to a material which cannot be physically removed in substantially one piece from a supporting substrate upon which it was placed. Related to degradation time, the weight loss of the material is another property that may be measured over time. In some embodiments, the material exhibits a desired weight loss (e.g., about 1%, about 5%, about 10%, about 15%, about 20%, about 25%, about 30%, about 40%, about 10% to about 40% or about 10% to about 30%) at a particular time point (e.g., at about seven days or about fourteen days) as measured relative to an initial time point (e.g., about 24 hours). Thus, the amount of soy protein and the amount of crosslinker used in the disclosed slurries for forming such a material may be those which achieve the desired weight loss. Similarly, the amount of soy protein, the amount of fiber-forming molecules, the weight ratio of the soy protein to the fiber-forming molecules and the molecular weight of the fiber-forming molecules used in the disclosed first solutions for forming such a material may be those which achieve the desired weight loss. Similarly, the molar ratio of the crosslinker to functional groups on the soy protein used in the disclosed second solutions for forming such a material may be those which achieve the desired weight loss. The Examples below describe techniques for determining the degradation time and weight loss of a material.

Regarding cell proliferation, this property may be quantified using DNA quantitation as described in the Examples below. In some embodiments, a soy protein-based porous material seeded with cells exhibits a desired increase (e.g., at least about 2-fold, at least about 3-fold, at least about 5-fold, or at least about 7-fold) in DNA content over a certain period of time (e.g., at least about seven days or at least about fourteen days). Thus, the amount of soy protein and the amount of crosslinker used in the disclosed slurries for forming such a material may be those which achieve the desired increase. Similarly, the amount of soy protein, the amount of fiber-forming molecules, the weight ratio of the soy protein to the fiber-forming molecules and the molecular weight of the fiber-forming molecules used in the disclosed first solutions for forming such a material may be those which achieve the desired increase. Similarly, the molar ratio of the crosslinker to functional groups on the soy protein used in the disclosed second solutions for forming such a material may be those which achieve the desired increase.

Regarding cell integration, this property may be evaluated using the techniques described in the Examples below. This property may be quantified via the percentage of cells which are integrated into the pores of the soy protein-based porous material (e.g., at least about 20%, at least about 30%, at least about 40%, at least about 50%, at least about 60%, at least about 70%, at least about 80%, or at least about 90%) as measured at a particular time point (e.g., at about seven days or about fourteen days). Thus, the amount of soy protein and the amount of crosslinker used in the disclosed slurries for forming such a material may be those which achieve the desired percentage. Similarly, the amount of soy protein, the amount of fiber-forming molecules, the weight ratio of the soy protein to the fiber-forming molecules and the molecular weight of the fiber-forming molecules used in the disclosed first solutions for forming such a material may be those which achieve the desired percentage. Similarly, the molar ratio of the crosslinker to functional groups on the soy protein used in the disclosed second solutions for forming such a material may be those which achieve the desired percentage. In some embodiments, the cells are integrated into the pores of the soy protein-based porous material and are substantially uniformly distributed throughout the material. Materials integrated with cells in this way may be distinguished from materials which may have certain regions which include cells integrated into the pores of the material and regions which do not include integrated cells.

The porous soy protein-based materials, tissue growth scaffolds comprising the materials and the methods of forming and using the materials will be understood more readily by reference to the following examples, which are provided by way of illustration and are not intended to be limiting.

### EXAMPLES

### Example 1

### Methods and Materials

Scaffold Fabrication

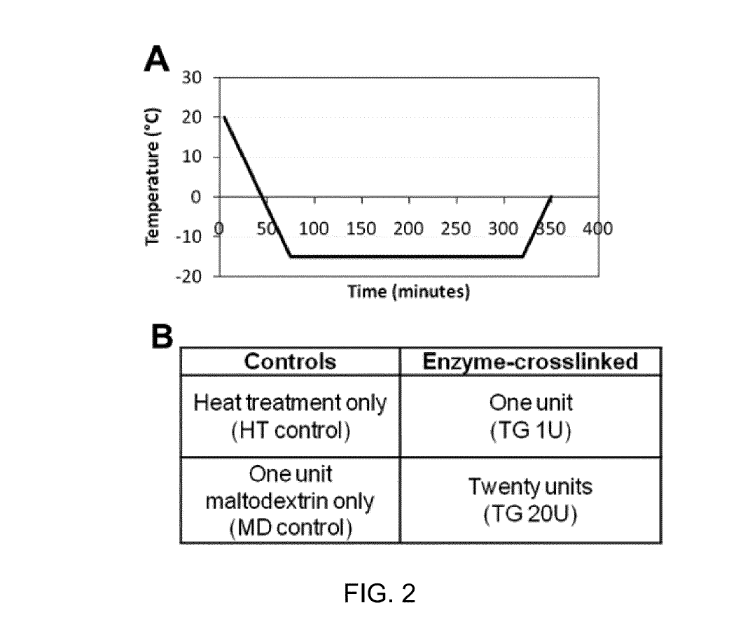

Soy protein isolate (SPI) containing approximately 83% pure soy protein (as verified by bicinchoninic acid (BCA) analysis from Thermo Fisher Scientific (Rockford, Ill., USA)) was obtained from Now Sports (Bloomingdale, Ill., USA). Mixtures of 3 weight percent (wt %) and 5 wt % SPI were dissolved in millipore water and were homogenized at 5,000 rpm for 5 minutes. The slurries were heated at 90° C. for 1 hour. Upon cooling of the solutions to room temperature, glycerol from Sigma-Aldrich (St. Louis, Mo., USA) was added in the same weight percent as SPI. The slurry was homogenized again at 5,000 rpm for 5 minutes. Slurries were casted in 7 cm diameter aluminum weigh boats at volumes of 18 mL and 13 mL for 3 wt % and 5 wt % SPI solutions, respectively. ACTIVA TI transglutaminase containing maltodextrin from Ajinomoto (Fort Lee, N.J., USA) was added to individual slurries in one and twenty units of enzyme activity to one gram of SPI. The added ACTIVA TI formulation included 1 wt % transglutaminase and 99 wt % maltodextrin. Maltodextrin (DE=4.0 to 7.0) from Sigma-Aldrich (St. Louis, Mo., USA) was added in the same weight to gram protein percentage as one unit transglutaminase as separate control samples. The slurries were incubated at 37° C. for 1 hour and then freeze-dried using a VirTis AdVantage BenchTop lyophilizer (Gardiner, N.Y.). The slurries were ramp frozen from 20° C. to −15° C. at a rate of 0.5° C./min, and the freezing temperature was held constant at −15° C. for 5 hours (FIG. 2). Frozen slurries were subsequently dried at 0° C. at a pressure of 100 mtorr for at least 40 hours to create a porous scaffold structure.

Materials Characterization

Scaffold Microstructure and Porosity:

Both dry and water-hydrated scaffolds were imaged using scanning electron microscopy (SEM). Dry scaffolds were desiccated for at least 2 hours prior to imaging. Hydrated scaffolds were first dehydrated in 95% ethanol for 3 hours then rinsed in water for 30 minutes, and the scaffolds immersed in water were lyophilized prior to imaging. Cross sections of all scaffolds were obtained through liquid nitrogen fracture and coated with 9 nm of osmium. SEM was performed using a LEO Gemini 1525 FEG SEM with an acceleration voltage of 15 kV (Oberkochen, Germany) to observe the scaffold microstructure. Mercury intrusion porosimetry from Micromeritics (Norcross, Ga.) was used to determine the volume percent porosity for all groups (n=3) using a previously described method. (Pappacena K, Gentry S, Wilkes T, Johnson M, Xie S, Davis A, et al. Effect of pyrolyzation temperature on wood-derived carbon and silicon carbide. J Eur Ceram Soc 2009; 29:3069-77.)

Moisture Content:

Moisture retention in scaffolds was analyzed immediately upon fabrication (n=5). Scaffold moisture content was determined for discs of 12 mm in diameter using a previously described method. (Reis R, Cohn D. Polymer based systems on tissue engineering, replacement and regeneration. Dordrecht, Netherlands: Kluwer Academic Publishers, 2002. p. 93-110.) Briefly, the initial weight upon casting (W₀) and weight after drying under vacuum at 40° C. for 24 hours (W_{d}) was used to calculate the moisture content (MC) using the formula MC=[(W₀−W_{d})/W₀]×100, immediately after lyophilization.

In Vitro Degradation:

Scaffolds were dehydrated in 95% ethanol for 3 hours and rinsed for 30 minutes in phosphate buffered saline (PBS) containing calcium and magnesium from HyClone (Logan, Utah, USA). 7 mm diameter scaffolds (n=4) were punched and subsequently weighed after lyophilization, then incubated at 37° C. in PBS or phenol red free DMEM media supplemented with 10% fetal bovine serum (FBS) and 1% antibiotic/antimycotic solution all from Invitrogen (Carlsbad, Calif., USA). Scaffolds were qualitatively checked for robustness with forceps every 3 days.

Compressive Mechanical Properties:

Compression testing was performed using a mechanical tester from JLW Instruments (Chicago, Ill., USA). Scaffolds were punched using a 7 mm biopsy punch, and the diameter was measured 3 times using calipers and averaged for calculation of stress. Samples were compressed at 0.2 mm/min up to 45% strain, with a preload of 0.1 N. The scaffold compressive modulus was determined by calculating the best fit slope of the linear (elastic) regime starting from the lowest strain outside of the toe region (0.8% strain) to R²=0.9. An average compressive modulus was determined for all scaffolds (n=4-5 per group). Both dry and water-hydrated scaffolds (prepared by 3 hours of 95% ethanol crosslinking and 30 minutes of rinsing in water) were tested.

In Vitro Cell Culture

Cell Seeding:

Human mesenchymal stem cells (hMSCs, Lonza) were grown in hMSC basal media containing mesenchymal stem cell growth supplement, L-glutamine, and penicillin/streptomycin all from Lonza (Walkersville, Md., USA). Cells were grown to approximately 90% confluence and trypsinized with 0.05% trypsin-EDTA from Invitrogen (Carlsbad, Calif., USA). Passage 5 cells were used for seeding. Scaffolds were sterilized in 95% ethanol for 3-4 hours and rinsed 3 times with PBS. The samples were briefly dried after the third rinse on 11 μm filter paper and immediately incubated at 37° C. for at least 30 minutes prior to seeding. hMSCs were top seeded with 200,000 cells per scaffold and allowed 10 minutes to adhere to the scaffolds at 37° C. before adding media to the wells. Seeded cells were cultured in phenol red free DMEM media supplemented with 10% fetal bovine serum (FBS) and 1% antibiotic/antimycotic solution all from Invitrogen (Carlsbad, Calif., USA) in a 37° C., 5% CO² humidified environment.

Scaffold Degradation:

Scaffolds were harvested at days 1, 7, and 14. At each harvested time point, weights of the seeded scaffolds were determined after lyophilizing the samples. The average of day 1 scaffold weights was considered the original scaffold weight with a relative weight fraction of 1. Relative weight fractions for the other two time points were calculated by dividing lyophilized scaffold weights by the average scaffold weight acquired from day 1.

Cell Morphology and Viability

At weekly time points, cell morphology and viability studies were performed using chemicals all acquired from Invitrogen (Carlsbad, Calif., USA). Live/Dead analysis was performed using the Live/Dead assay kit staining live cells with calcein and dead cells with ethydium bromide. Stained cells and the protein scaffold were visualized using an LSM Meta 510 CLSM from Zeiss (Jena, Germany). DNA quantification was performed using a Picogreen assay kit following the manufacturer's protocol.

Statistical Analysis

The quantitative data were reported as mean±standard error mean (S.E.M.=standard deviation/n^{1/2}). One-way analysis of variance (ANOVA) was performed with the treatment group as the fixed factor. P-values less than 0.05 were considered statistically significant. Two-way ANOVA was performed for DNA quantification data, where the two fixed factors were group and time point. Independent t-tests assuming unequal variance were performed between each combination of treatment groups to reduce the likelihood of type I errors.

Results

Scaffold Microstructure and Porosity

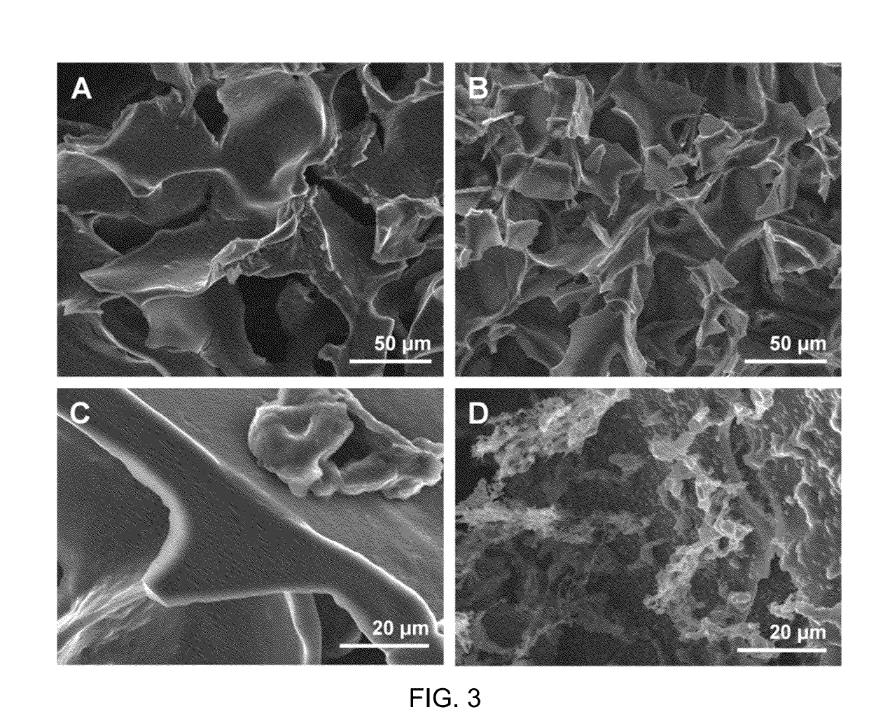

The thickness of all 3% and 5% SPI scaffolds ranged from 1.7 to 1.9 mm and 2.2 to 2.4 mm respectively. Observations of scaffold microstructure using scanning electron microscopy revealed rough surfaces of scaffold struts (FIG. 3A-D). All scaffolds exhibited irregular pore shapes. Struts of non-hydrated structures appeared thicker compared to hydrated scaffolds. The struts of hydrated scaffolds were thinned along the edges. In scaffolds with twenty units of enzyme crosslinking, precipitates formed on the surfaces of the protein struts. This phenomenon was not observed in the one unit crosslinked or one unit maltodextrin-only scaffolds.

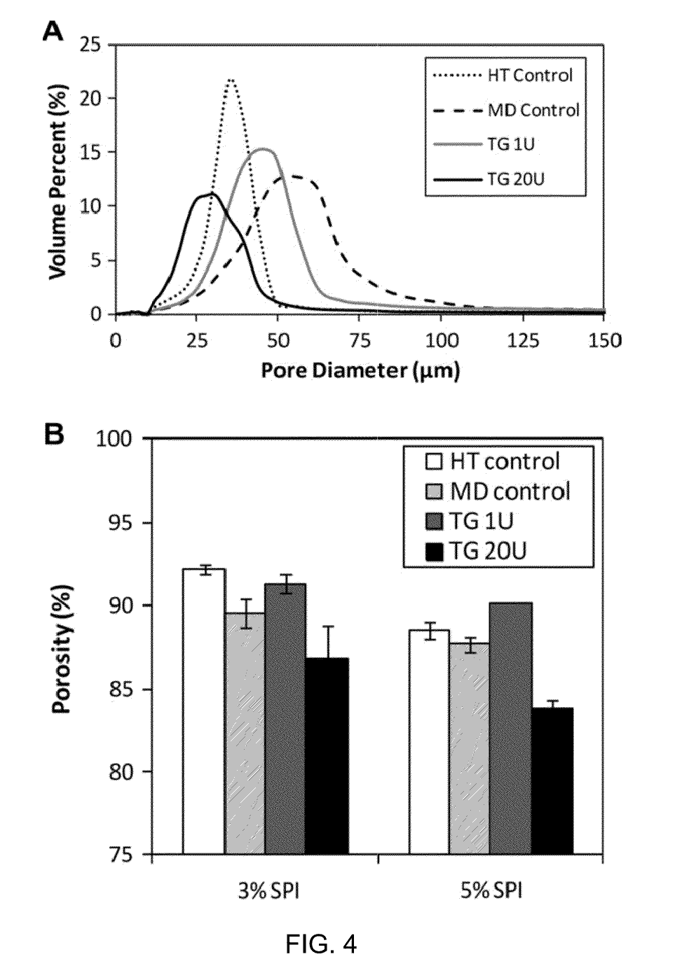

All scaffold pore sizes were normally distributed in the range of 10 to 150 μm (FIG. 4A). Both 3% and 5% scaffolds followed the same distribution trend with varying treatments. The cumulative volume percent of pore sizes ranging from 150-1000 μm was less than 4%. Maltodextrin-only scaffolds yielded a higher median pore diameter. The pore size distribution was wider for scaffolds containing maltodextrin compared to the HT control scaffolds. The median pore diameter decreased with increased enzymatic crosslinking. Total porosity of scaffolds was consistently greater than 80%, with no significant difference as protein content increased (FIG. 4B). The 5% SPI twenty unit enzyme-treated group had significantly lower porosities compared to all other groups (p<0.01) except when compared to the similar 3% group, and one unit of enzyme treatment in the 5% SPI scaffolds resulted in significantly different porosity compared to the one unit maltodextrin-only control (p<0.05).

Moisture Content

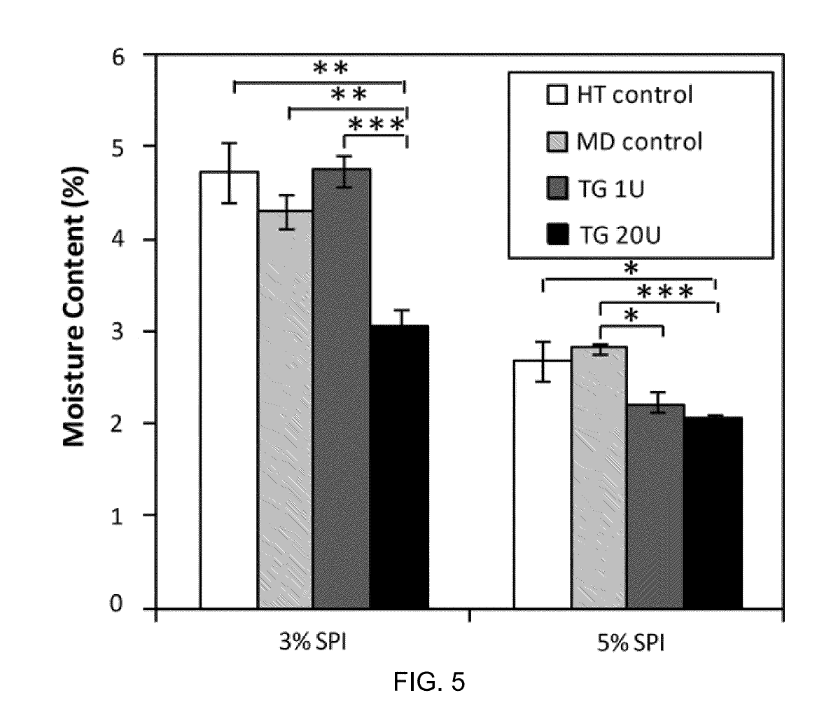

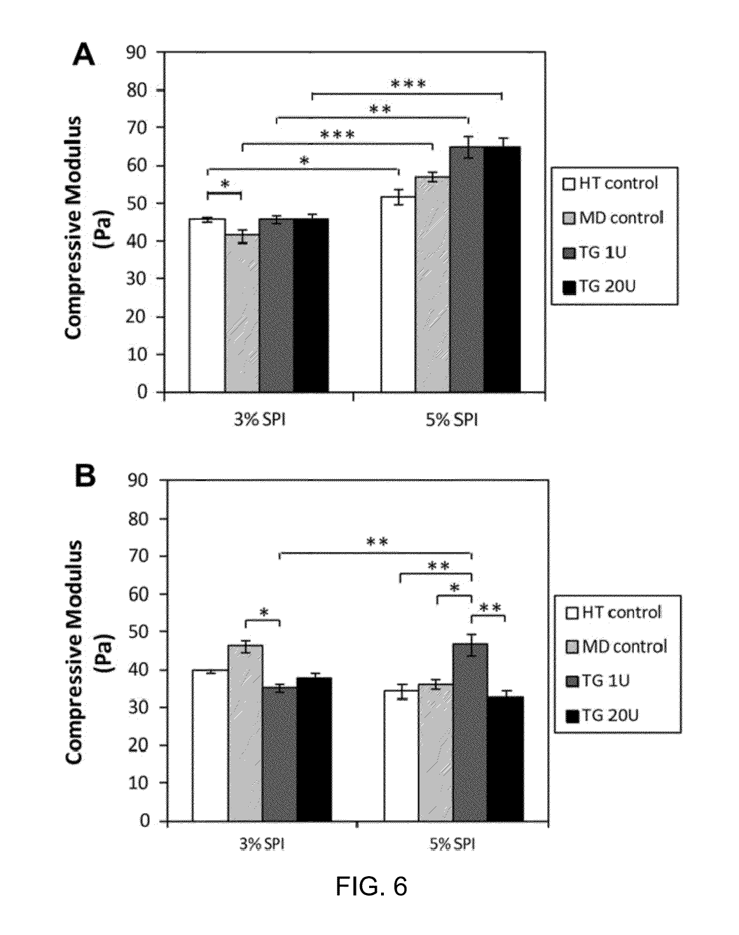

Scaffolds retained 2-5% moisture content (FIG. 5). One-way ANOVA demonstrated that increasing the percentage of soy from 3% to 5% significantly decreased the moisture content for every treatment (p<0.005). The 3% SPI scaffold crosslinked with twenty units of transglutaminase had a significantly lower moisture content compared to all other 3% SPI groups (p<0.005). Although increasing the amount of enzymatic crosslinking significantly decreased moisture content for 3% SPI scaffolds, this effect was not significant for 5% SPI scaffolds.

Compressive Mechanical Properties

Scaffolds under compression demonstrated viscoelastic behavior. Compressive moduli for dry scaffolds within each treatment group increased as the protein content increased from 3% to 5% (FIG. 6A). Enzyme-treated groups significantly increased the modulus for 5% SPI scaffolds only (p<0.05). No difference in modulus was observed when the number of enzyme units applied was increased. Upon hydration, the compressive modulus significantly decreased for all 5% SPI groups (p<0.001) and for the 3% SPI twenty unit enzyme-treated group (p<0.005) (FIG. 6B). The 3% maltodextrin control scaffolds had higher moduli compared to enzyme-treated scaffolds when hydrated. The 5% SPI one unit enzyme-treated scaffolds had the highest moduli compared to all other 5% SPI groups.

Cell Growth and Scaffold Degradation

Qualitative observations of scaffold robustness using forceps to maneuver the structure revealed that scaffolds soften when hydrated, yet remain intact. Non-seeded scaffolds decomposed over time into many fragments that were not recoverable or able to be lifted out of the well. Comparisons across scaffold groups between the first time points at which scaffolds could not be recovered showed that in general, scaffolds degraded faster in PBS compared to media, except for the 3% SPI heat treatment-only control group (see Table 1, below). Cells seeded onto scaffolds allowed the scaffolds to remain intact for a longer period of time. Enzyme treatment increased time to complete degradation for both 3% and 5% SPI scaffolds, especially for scaffolds in media. Cell-seeded scaffolds remained intact longer with enzyme treatment compared to control groups for 3% SPI scaffolds.

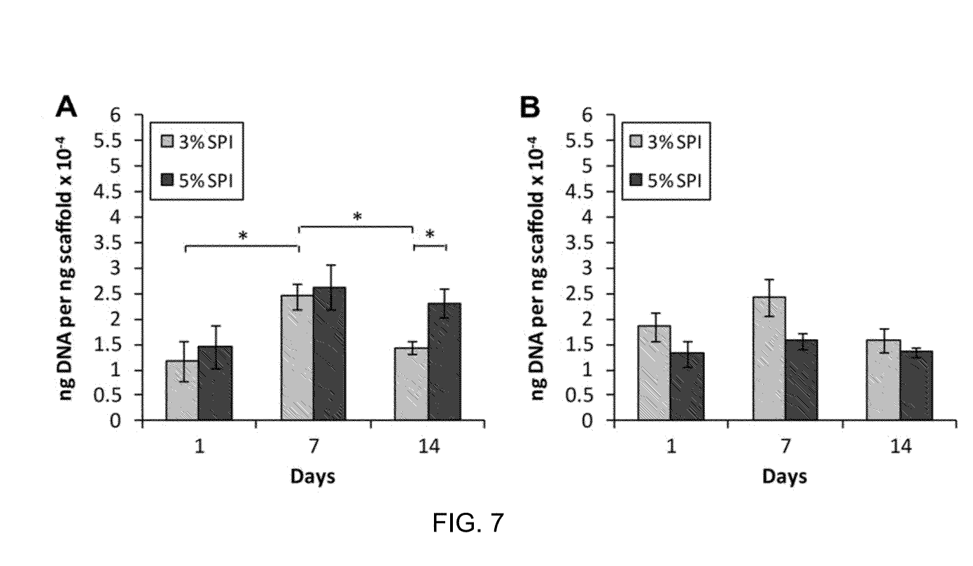

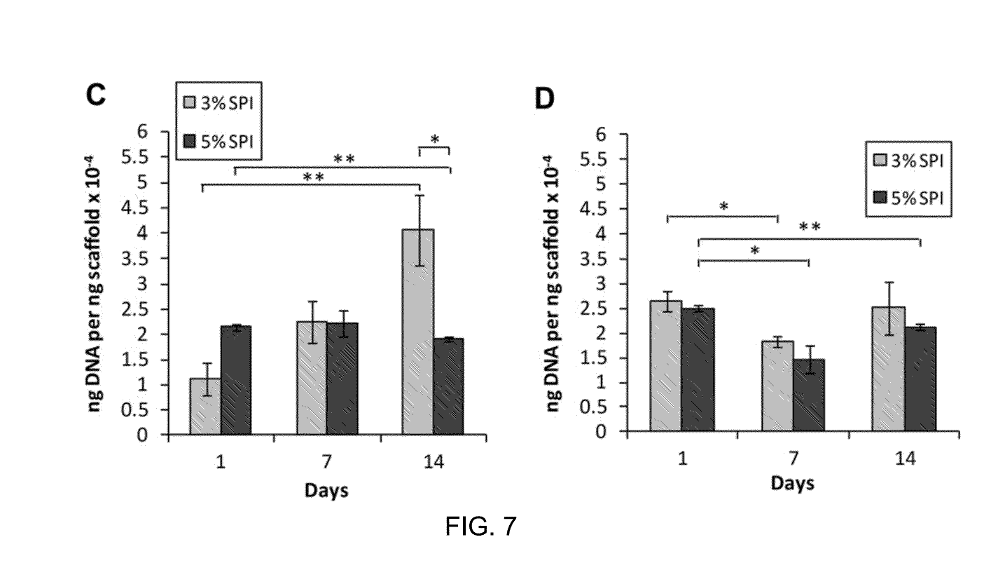

All scaffolds supported cell attachment and cell viability for fourteen days (FIG. 7A-D). In the heat treatment control group, the amount of DNA significantly increased from day 1 to day 7 and then decreased at day 14 for the 3% SPI scaffolds. At day 14, the amount of DNA for this group was significantly less for 3% SPI scaffolds compared to 5% SPI scaffolds. There was no significant change in DNA content for the maltodextrin-only scaffolds. In the one unit enzyme-treated group, cells proliferated for the 3% SPI scaffolds, with a 3 fold increase in DNA content over fourteen days. The DNA content was constant over fourteen days for the 5% SPI scaffolds. At day 14, the amount of DNA for one unit enzyme-treated group was significantly higher for the 3% SPI scaffolds compared to 5% SPI scaffolds. In the twenty unit enzyme-treated group, DNA content decreased from day 1 to day 7 and increased from day 7 to day 14 for both 3% and 5% SPI scaffolds.

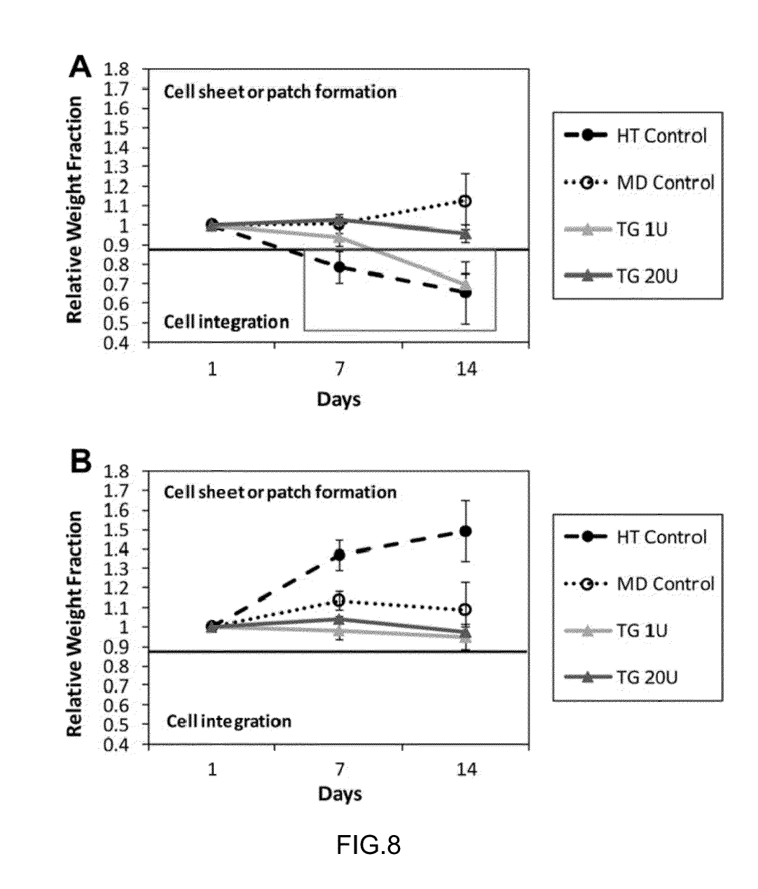

Fluorescence microscopy images of the surfaces of scaffolds were obtained with Live/Dead staining along with confocal fluorescence three-dimensional reconstruction of the scaffold surface (data not shown). Cell morphology changed over time as cell growth occurred on and into the surface of the scaffolds. Blank scaffolds did not yield any Live/Dead fluorescence signal upon staining. However, protein autofluorescence remained visible upon UV excitation. Observations of 3% SPI scaffolds showed that cells either formed a cell sheet or integrated into the scaffold pores. Cells were spread thinly on the protein scaffold for both control groups (HT and MD groups) at days 1 and 7 but formed cell layers at day 7 for the one unit enzyme-treated group and at all time points for the twenty unit enzyme-treated group. After fourteen days, the cells had integrated into the pores for the heat treated control group and the one unit enzyme-treated group. A layer of cells covering the outer surface of the scaffold was observed on both the maltodextrin control group and the twenty unit enzyme-treated group. The confluent cell sheet appeared denser for the twenty unit enzyme-treated group compared to all other cell sheets that were observed. Comparison of the relative weight fractions of the scaffolds with the Live/Dead images showed that scaffolds that had degraded more than 10% from their original weight at day 1 resulted in cells integrating within the scaffold (FIG. 8A-B). Scaffolds that had minimal degradation of less than 10% were more stable and resulted in cells forming a cell sheet across the scaffold surface. The 5% SPI scaffolds maintained integrity with less than 10% weight loss over fourteen days. These samples contained cells with elongated and aligned structures similar to the 3% SPI scaffolds that had the same degradation pattern (data not shown).

Discussion

Although soy protein is globular, heat treatment applied to protein slurries was able to induce interconnected, porous, three-dimensional scaffolds upon freeze-drying. Protein chains relax upon heating, and a degree of denaturation allows proteins to entangle in solution. The denaturation temperature of the 75 and 11S subunits is dependent on the water content of the slurry. Higher water content and lower protein concentration decreases the denaturation temperature of both subunits; the ideal denaturation temperature that avoids decomposition has been reported as 90° C. (Zhang H, Takenaka M, Isobe S. DSC and electrophoretic studies on soymilk protein denaturation. J Therm Anal Calorim 2004; 75:719-26.) Glycerol and water were added as plasticizers to decrease the slurry glass transition temperature, which increased the elasticity of the resulting freeze-dried scaffold. The range of pore sizes in hydrated scaffolds is well defined, and moisture content is in the same range (5% moisture) as previously reported soy protein materials. (Vaz C, Fossen M, van Tuil R, de Graaf L, Reis R, Cunha A. Casein and soybean protein-based thermoplastics and composites as alternative biodegradable polymers for biomedical applications. J Biomed Mater Res A 2003; 25:60-70.) Although the mechanical strength is on the order of Pa, all scaffolds were robust enough to support cell attachment, and strength did not significantly diminish when scaffolds were in the hydrated state. Differences in hydrated compressive moduli between groups can be attributed to protein struts on the scaffold surface dissolving away from the scaffold during compression. Degradation of protein scaffolds in PBS occurred faster than in media due to the differences in electrostatic interactions. Salts in PBS can interact with the negatively charged soy protein to break up amino acid chains, while proteins in media may adhere to the scaffold and provide resistance to degradation. When cells were present, attached cell processes acted like crosslinks and pulled protein struts together, which increased degradation time for all scaffold groups and helped maintain scaffold structural integrity over time.

Maltodextrin and transglutaminase did not greatly widen the range of the compressive strength and moisture content in the soy protein scaffolds. However, the additives had a significant effect on pore size distributions, degradation properties, and biological activity of seeded cells. From the resulting SEM images, maltodextrin appeared to have leeched from the protein scaffold walls, since attached particles are seen on the walls of the hydrated scaffolds (FIG. 3A-D). The precipitates were present in large quantities in the twenty unit scaffolds and in smaller, yet visible amounts in the one unit maltodextrin and transglutaminase scaffolds but were not observed in the heat treatment only scaffolds. Increased amounts of maltodextrin decreased degradation times for scaffolds due to leeching of maltodextrin from the protein struts, but the degradation time was slowed by enzyme crosslinking. Enzyme treatment successfully increased the degradation time of the scaffolds; scaffolds containing transglutaminase with maltodextrin degraded slower than scaffolds containing the same amount of maltodextrin only (see Table 1, below).

TABLE 1
Scaffold degradation time, determined qualitatively by observing
robustness of scaffolds at various time points. Days above are
the first observed time point where at least one scaffold in
the group has completely degraded. Blank scaffolds in PBS and
media (diameter = 7 mm) were observed every three days
in culture (n = 4), while scaffolds with cells (diameter =
4 mm) were observed weekly (n = 4-5).

| | | Bare | Bare | Cell-seeded |
|---|---|---|---|---|
| | | scaffolds | scaffolds | scaffolds |
| | | in PBS | in media | in media |
| | Group | (days) | (days) | (days) |
| 3% | HT Control | >14 | 4 | 14 |
| SPI | MD Control | 4 | 4 | 14 |
| | TG 1U | 7 | >14 | >14 |
| | TG 20U | 11 | >14 | >14 |
| 5% | HT Control | 7 | 7 | >14 |
| SPI | MD Control | 4 | 7 | 14 |
| | TG 1U | 4 | 14 | >14 |
| | TG 20U | 7 | >14 | >14 |

Cell growth behavior was different across all scaffold groups. Heat-treated control scaffolds resulted in sparse cell integration. An explanation is that proliferation was not observed for both heat treated and maltodextrin control groups because the scaffold proteins degraded faster in the media than the cells can grow, as indicated by degradation times of seven days or less for non-seeded scaffolds in media (see Table 1, above). In this case, the scaffold was only robust enough to sustain cell viability over fourteen days. A cell layer formed for the maltodextrin control scaffolds even though no proliferation was observed; this may be due to cellular digestion of the scaffold and the interaction of cells with the maltodextrin chains. Proliferation was not observed in twenty unit enzyme-treated scaffolds, even though cell sheets formed at early time points and the presence of greater amounts of maltodextrin could have increased cell metabolism. Since these scaffolds were stable and robust, degradation was limited. Cells can only spread on the surface until full confluence is reached. There were a greater number of cells present at earlier time points for this group as observed in Live/Dead staining, indicating that cells reached confluence faster compared to other groups, possibly due to greater concentration of added maltodextrin. Cell proliferation and integration in the 3% SPI one unit enzyme-treated group were observed perhaps due to a balance of scaffold robustness, degradation time, and presence of maltodextrin. Strut relaxation and degradation over time may have allowed surrounding nutrients to better penetrate and flow through the pores to stimulate cell growth on and within the scaffold.

### Example 2

### Materials and Methods

Commercial soy protein isolate (protein content >90%) from Now Foods, Ill., USA was used without further purification for all of the electrospinning experiments. PEO with an average molecular weight of 1×10⁵, 1×10⁶ and 8×10⁶ Da, sodium hydroxide (NaOH), 1-Ethyl-3-(dimethylaminopropyl)-carbodiimide (EDC), N-hydroxysulfosuccinimide (NHS), glutaraldehyde and sucrose were purchased from Sigma-Aldrich, USA.

SPI/PEO Solution Preparation.

Various concentrations of SPI, PEO and 1% NaOH (wt/wt) were homogenized in an ice bath for 10 min at 20,000 rpm using IKA ultra-turrax T18 basic homogenizer and S18N-10G mixer. The solution was heat treated at 60° C. in a water bath for 2 h. The solution was cooled down to room temperature (20±1° C.) before electrospinning.

Electrospinning.

The SPI/PEO solution was placed in a syringe and mounted on a syringe pump (Model 100, KD Scientific Inc., USA). The syringe was capped with a 20-gauge blunt end needle. The solution was delivered through a Teflon tube (Cole-Parmer, USA) at a constant flow rate to the 20-gauge blunt end needle connected on the other end. The lead of a high voltage supply (Model ES30, Gamma high voltage research Inc., Florida, USA) was connected to this needle. A collector made up of aluminum was connected to the ground of the high voltage supply. Upon applying positive voltage, a charged jet of SPI/PEO solution was ejected from the tip of the needle depositing on a piece of aluminum foil affixed to the grounded collector to form a nonwoven fiber mat. The solution flow rate and working distance between needle tip and collector were optimized to 0.004 ml/min and 18 cm, respectively.

EDC/NHS Crosslinking.

EDC and NHS were dissolved in 100% ethanol (Columbus Chemical Industries Inc., USA) at the molar ratio of 10:4:1 EDC and NHS to COOH. Electrospun mats were crosslinked by immersing them in EDC/NHS solution for 24 h.

Surface Tension and Viscosity Measurements.

Surface tension of SPI/PEO solutions were measured using a drop shape analysis system (DSA100, KRUSS Advanced Surface Science, Germany) with a 14 gauge blunt tip needle (Nordson EFD, USA). Viscosity of SPI/PEO solutions was determined using Anton Paar Physica MCR 300 modular compact rheometer. Viscosity was measured at shear rates of 1 through 100 s-1 at 25° C.

Scanning Electron Microscopy (SEM) and Fiber Diameter Measurements.

SPI/PEO electrospun fiber morphology and hMSC attachment and spreading on electrospun scaffolds were observed using scanning electron microscopy (Hitachi S-4800-II) at an accelerating voltage of 3 kV after sputter coating with osmium to create a 9 nm thick coating. Average fiber diameter was determined from SEM images by taking at least 25 measurements using an image analysis software INCA. For the average fiber diameter measurements, areas with beads and spindles were ignored.

Tensile Test.

Electrospinning of 7% SPI/3% PEO (1MDa) and 12% SPI/3% PEO (1MDa) solutions were carried out for 1.3 h to produce electrospun mats. Dog-bone shaped specimens of 47×3×t mm (length×width×thickness) were cut from SPI/PEO electrospun mats and were crosslinked using 10 mM EDC/NHS. After crosslinking electrospun scaffolds were hydrated for tensile testing. The dimensions of the specimens were measured using a digital caliper. Tensile tests were performed at room temperature with LFPlus mechanical tester integrated with NexygenPlus software (Loyd Instruments Ltd, UK). Tests were performed using a cross-head speed of 3 mm/min and a loading cell of 0.002 kN. The electrospun samples were stretched to failure.

Human Mesenchymal Stem Cell Culture.

Human mesenchymal stem cells were purchased from Lonza Walkersville, Inc., MD (# PT-2501). Cells were cultured in MSCGM Basal Medium (Lonza Walkersville, Md.) with 10% fetal bovine serum (FBS), 2 mM of L-glutamine and 100 IU/ml penicillin-streptomycin. Electrospinning of 7% SPI/3% PEO (1MDa) and 12% SPI/3% PEO (1MDa) solutions were carried out for 1.3 h to produce electrospun mats for cell studies. Electrospun mats were punched out into circular shape scaffolds with 6 mm diameter. These scaffolds were crosslinked using EDC/NHS and washed thoroughly with 100% ethanol. Before cell seeding, the scaffolds were washed subsequently twice in 70% ethanol and three times in 1× DPBS buffer (Hyclone) each for 10 min. The scaffolds were sterilized under UV radiation for 30 min before seeding the cells. Passage 5 hMSCs were seeded on SPI/PEO electrospun scaffolds at a density of 50,000 cells/well. Cells were cultured in humidified 5% CO₂ incubator kept at 37° C. Cell culture media was exchanged every 3-4 days. Cell viability and spreading were studied on days 1 and 7 of culture.

Cell Viability Using Live/Dead Stain.

Cell viability was assessed using a live/dead assay kit (Invitrogen # L-3224). The staining solution was prepared by adding 9.5 μl of ethidium homodimer-1 to 2 ml DPBS, followed by 4.6 μl calcein AM. The media was aspirated from the cell wells, and scaffolds were washed gently with 1× DPBS (Hyclone). 100 μl of live/dead stain solution was added per well and incubated for 20 min at 37° C. The scaffolds were removed from cell culture well plates and placed upside down on a glass bottom microwell dish (MatTek Corporation, MA) for imaging. Separate live and dead fluorescence images were taken using mercury lamp (CHIU Technical Corporation) Nikon Eclipse TE 2000-U microscope in conjunction with blue (450-490 nm) and green (510-560 nm) filters. The color images were then merged using Metaview imaging software.

PicoGreen dsDNA Quantization.

Total dsDNA content in each scaffold was determined using Quant-iTTM PicoGreen dsDNA assay kit (Invitrogen). The scaffolds were incubated overnight in a water bath at 37° C. after adding 100 mg/ml Protienase K in Tris-HCl buffer solution to digest the scaffolds. dsDNA quantization was carried out according to the manufacturer's protocol using a Spectramax M5 microplate reader (Molecular Devices Corporation, CA).

Cell Morphology Analysis Using SEM.

Cell attachment and spreading on SPI electrospun scaffolds was investigated using SEM. The scaffolds were fixed in 2% glutaraldehyde and 3% sucrose for 2 h. After fixing, scaffolds were dehydrated in sequential ethanol series (70%, 80%, 90% and 100%). Scaffolds were critically point dried using a critical point dryer (Tousimis Research Corporation, Md.). Scaffolds were sputter coated with osmium (9 nm) prior to SEM analysis.

Results

Influence of Applied Voltage.

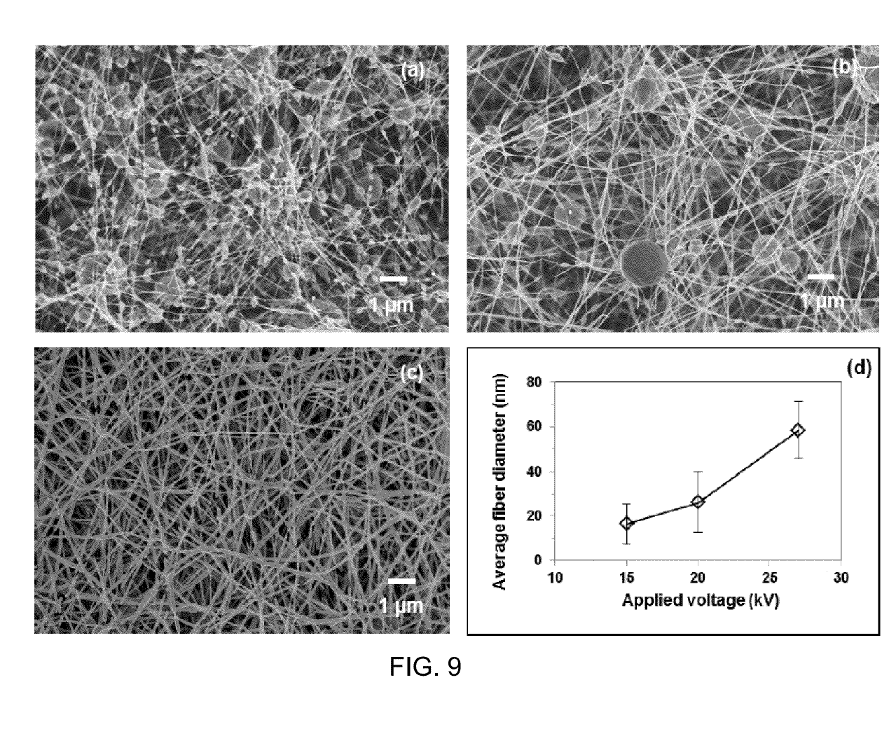

The applied voltage during the electrospinning process was optimized to obtain continuous and uniform electrospun fibers from SPI. FIG. 9A-D shows the influence of applied voltage on electrospun fiber morphology of 7% SPI/5% PEO (100 kDa). Droplets with short linear fibers were formed at an applied voltage of +15 kV. Longer linear fibers with beads and spindles were produced by increasing the applied voltage to +20 kV. The solution could be successfully electrospun into continuous fibers with minimum defects by further increasing the applied voltage to +27 kV. The longer linear jet, which resulted in the formation of continuous fibers, was produced due to higher electrostatic repulsion force between the needle tip and collector with increasing applied voltage, which in turn provides higher drawing stress on the jet. The average fiber diameter was found to increase with increasing applied voltage. The applied voltage may affect the mass of polymer fed out from tip of the needle, elongation and splaying of the jet. Therefore, it can be concluded that the increased electrostatic force with increasing applied voltage draws more SPI/PEO solution out of the needle that results in an increase in average fiber diameter.

Change in Electrospun Morphology with SPI and PEO Concentrations.

Table 2 shows the electrospinnability of various SPI/PEO solutions investigated and their electrospun morphology. Even though the mixture of SPI and PEO could successfully form fibers, neither SPI nor PEO alone at the chosen concentrations were able to form electrospun fibers. Prior to electrospinning, SPI/PEO solutions were prepared by heat treating them at 60° C. in alkaline conditions (pH 13) for 2 h. While SPI was unfolded and hydrolyzed during the heat treatment, PEO which has both hydrophilic ether oxygen and hydrophobic methylene segments interacts with the amino acids of SPI protein through ionic, hydrogen and hydrophobic interactions. These interactions between SPI and PEO may explain the ability to electrospin mixtures of the two solutions and not the individual polymer solutions.

TABLE 2
Electrospinnability of SPI/PEO solutions

| Solution chemical | |
|---|---|
| composition | Electrospun morphology |
| 7% SPI | Droplets |
| 5% 100 kDa PEO | Droplets |
| 5% SPI/5% 100 kDa PEO | Short fibers with droplets |
| 7% SPI/5% 100 kDa PEO | Continuous fibers with beads and spindles |
| 10% SPI/5% 100 kDa PEO | Continuous fibers with lesser beads and |
| | spindles |
| 12% SPI/5% 100 kDa PEO | Continuous fibers |
| 7% SPI/10% 100 kDa PEO | Fibers with lesser beads and spindles |
| 7% SPI/15% 100 kDa PEO | Continuous fibers with lesser beads and |
| | spindles |
| 7% SPI/20% 100 kDa PEO | Continuous fibers |
| 7% SPI/3% 100 kDa PEO | Short fibers with droplets |
| 7% SPI/3% 1 MDa PEO | Continuous fibers |
| 7% SPI/3% 8 MDa PEO | Continuous fibers |

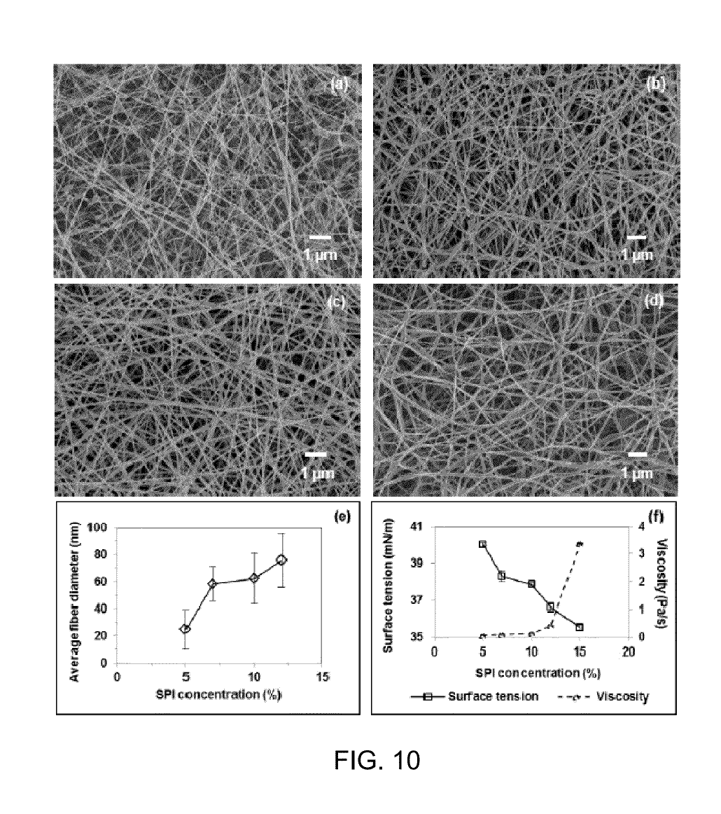

Typical fiber structures exhibiting the effect of SPI and PEO concentrations are shown in FIGS. 10A-F and 11A-F, respectively. The results revealed that electrospun fiber morphology was affected by both SPI and PEO concentrations. Beaded structures with short fibers were produced for solutions containing lower concentrations of either SPI or PEO. If the concentration of either SPI or PEO is increased, the structural features changed from beaded fibers with round beads to beaded fibers with spindle-like beads to more uniform fibers with lesser defects.

Increasing the concentration of SPI is beneficial in the formation of continuous uniform fibers due to denaturation of the protein during heat treatment of the SPI/PEO solution at high pH (pH 13). The adjacent portions of proteins start to repel each other due to high charge density at extreme alkali pH which leads to irreversible unfolding of protein molecules. These conditions make the soy protein remarkably stable against thermal aggregation. The refolding of protein molecules even at room temperature is inhibited at this high pH condition. The more the number of unfolded protein chains (i.e. higher concentrations of SPI), the easier the formation of continuous fibers during the electrospinning process. The fact that continuous fibers with lesser number of beads formed with increasing concentration of PEO (holding SPI concentration constant) indicates that PEO enhanced polymer chain entanglement, which can prevent the polymer jet from breaking up into droplets. These results therefore reveal that the electrospinnability of SPI/PEO solutions is possible due to the synergistic effect of unfolded hydrolyzed SPI protein chains and entanglement of PEO polymer chains.

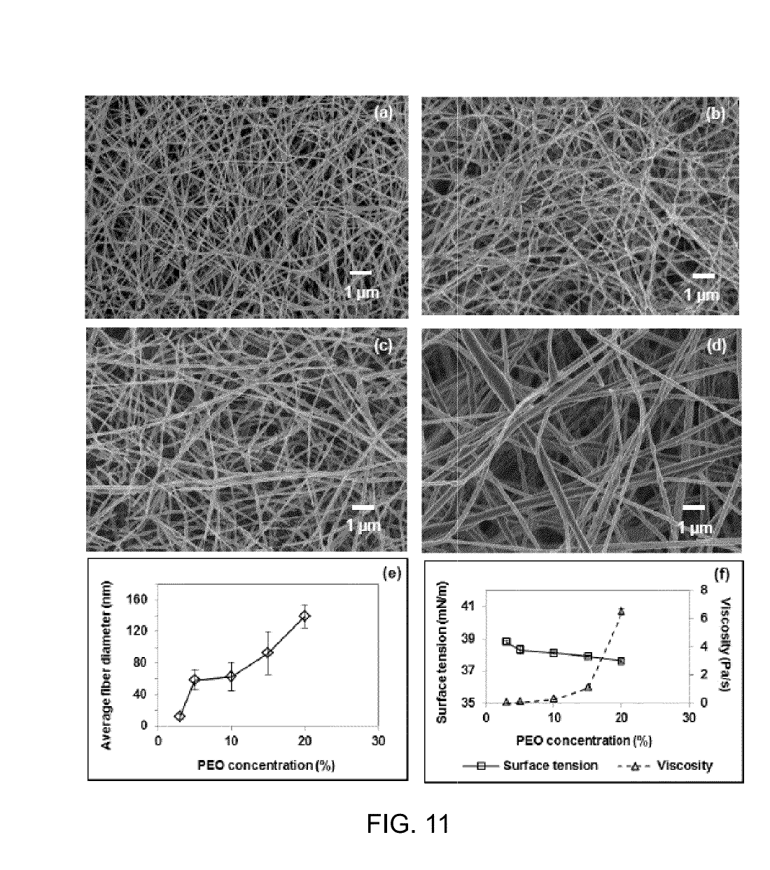

The solution surface tension and viscosity are factors that affect spinnability and morphology of electrospun fibers. FIGS. 10F and 11F include the surface tension and viscosity for the SPI/PEO solutions studied. A slight decrease in surface tension and a significant increase in viscosity were observed while increasing the concentration of either SPI or PEO. SEM images revealed the formation of continuous fibers with lesser defects for SPI/PEO solutions with lower surface tension and higher viscosity. In the case of lower viscosity solutions, surface tension plays a dominant role and thus beads or beaded fibers are formed due to the tendency of solvent molecules to congregate to form droplets under the action of surface tension. At higher viscosity, the interaction between protein and polymer was improved which reduced the action of surface tension and resulting in continuous fiber formation.

FIG. 10E shows the average fiber diameter plotted as a function of SPI concentration. The average fiber diameter increased from 30 to 90 nm with increasing SPI concentration. FIG. 11E shows the increase in average fiber diameter from 30 to 150 nm with increasing PEO concentration. The increase in average fiber diameter with increasing either SPI or PEO concentration is due to greater entanglement of macromolecules in the mixed solution, which increases the solution viscosity that causes the formation of larger fiber diameters. It has been shown that the fiber diameter depends allometrically on solution viscosity in the form, d∝η^{α}, where d is the diameter of the electrospun fiber, η is viscosity, and α the scaling exponent. Other experimental observations and theoretical analyses show that viscosity has a power relation with concentration, η∝C^{β}, where C is the solution concentration and β is the scaling exponent. The observations therefore coincide with these relationships since it was demonstrated that increasing the concentration of SPI or PEO led to an increase in fiber diameter.

In all SPI and PEO mixture formulations a second population of smaller size fibers with diameters ranging from 15 to 25 nm was observed due to the splaying of the polymer jet. During electrospinning, the diameter of the positively charged jet decreases while travelling in air due to the simultaneous effect of stretching of the jet and evaporation of the solvent. As the jet diameter decreases, the surface charge density increases. The high repulsive forces from the increased charge density split the jet and the smaller jets splay. The smaller jets then split and splay as their diameter is reduced further. This process may be repeated several times to create many small jets which dry rapidly to form fibers with very small diameter.

Influence of PEO Molecular Weight.

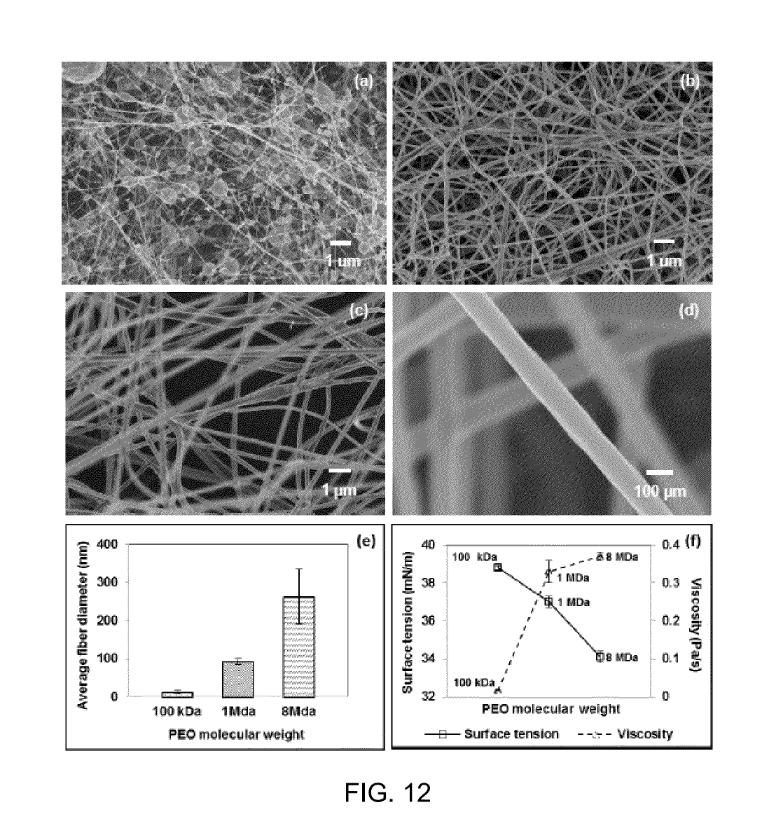

FIG. 12A-F exhibits the effect of PEO molecular weight on electrospun fiber morphology. The average fiber diameter and inter-fiber spacing of the SPI/PEO electrospun fibers increased significantly with increasing PEO molecular weight. During electrospinning, the charged jet ejected from the Taylor cone is subjected to tensile stresses and undergoes significant elongational flow. The nature of this elongational flow determines the degree of subsequent splitting and splaying of the jet. This elongational flow is dependent on the elasticity of the solution, which depends on the polymer molecular weight. It has been reported that the relaxation of the polymeric chain becomes more difficult as the molecular weight increases and the jet splitting and splaying processes are not as effective. As a result, the fiber diameter increases with increasing molecular weight as shown in FIG. 12E. It has been shown that the Trouton ratio of PEO (ηₑₓₜₑₙₛᵢₒₙₐₗ/ηₛₕₑₐᵣ) increased with increasing molecular weight. This increase in Trouton ratio may also reduce the extent of jet splitting and splaying and hence increases the fiber diameter in the presence of a higher molecular weight PEO.

The viscosity increased and surface tension decreased with increasing PEO molecular weight (FIG. 12F). Hence, continuous fibers with lesser defects were produced in the presence of higher molecular weight PEO. The increase in fiber diameter with increasing PEO molecular weight may be also due to the increased viscosity, which leads to more entanglement of the molecules.

Effect of 1-Ethyl-3-(dimethylaminopropyl)-carbodiimide/N-hydroxysulfosuccinimide (EDC/NHS) Crosslinking.

SPI/PEO electrospun scaffolds were crosslinked with EDC/NHS to enhance structural integrity and stability. EDC and NHS concentrations and duration of crosslinking were varied and better structural integrity of electrospun fibers was observed at a molar ratio of EDC to NHS to COOH of 10:4:1 after 24 h crosslinking. Crosslinking of SPI using EDC/NHS involves the activation of carboxylic acid groups present in the polypeptide chain followed by reaction with free amine groups of another polypeptide chain. The advantages of using this crosslinking method are that the crosslinking agent is not incorporated within the material and excess reagent can be easily removed by thorough washing.

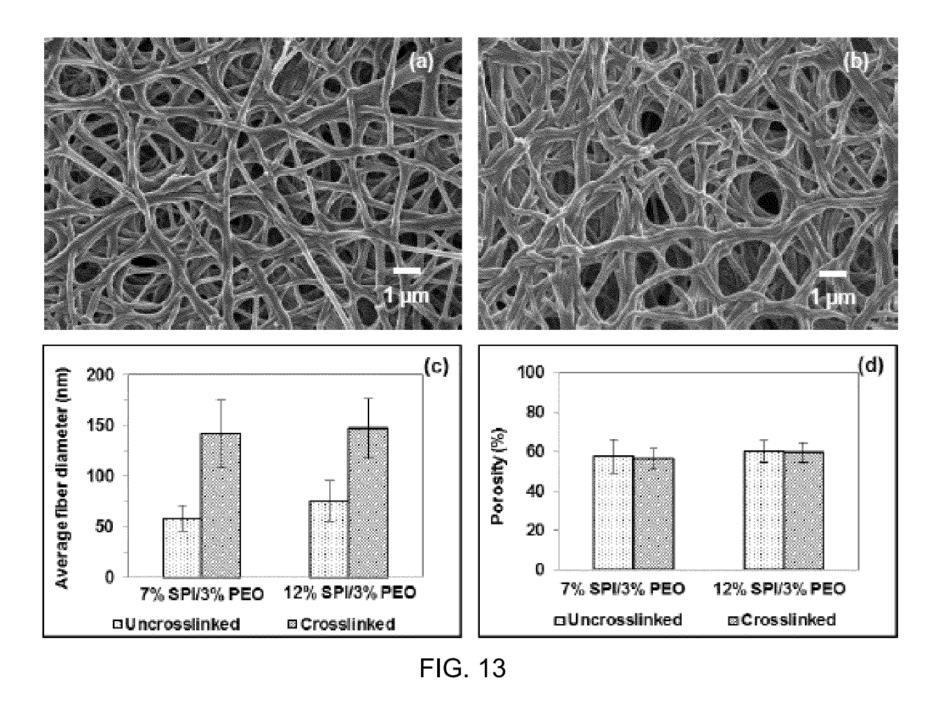

SPI electrospun morphology after EDC/NHS crosslinking is shown in FIG. 13A-D. SEM images indicate that the fibers bundle after the crosslinking treatment. The fibers of the uncrosslinked electrospun scaffolds stay separated because of their electrostatic repulsion, whereas the crosslinking treatment resulted in bundling of the fibers. The resulting diameters of the crosslinked and uncrosslinked electrospun fibers are presented in FIG. 13C. The fiber diameter significantly increased after crosslinking. The porosity of SPI electrospun scaffolds before and after crosslinking was measured by placing a grid on the SEM images and calculating the percentage of points that fall within the pores using ImageJ software. Points that fell on the boundaries of pores were counted as one half of a point. The counted points were divided by total number of points in the grid and multiplied by 100 to determine the % porosity. Results revealed that there was no significant decrease in porosity after crosslinking (FIG. 13D).

Tensile Properties of Crosslinked SPI Electrospun Scaffolds.

Table 3 shows the tensile properties of hydrated crosslinked 7% SPI/3% PEO and 12% SPI/3% PEO electrospun scaffolds (spun for 1.3 h). Young's modulus (E′) was found to be 110±6 and 171±21 kPa for 7% SPI/3% PEO and 12% SPI/3% PEO, respectively. The modulus for the 12% SPI/3% PEO scaffold was higher most likely due to higher soy protein content. A tensile strength of 0.06±0.01 and 0.17±0.006 MPa was observed for 7% SPI/3% PEO and 12% SPI/3% PEO, respectively. The higher tensile strength of the 12% SPI condition may be due to the higher protein content as well as the greater average fiber diameter (FIG. 10E). Results indicated that the strain at break were similar for both 7% SPI/3% PEO and 12% SPI/3% PEO conditions (Table 3).

TABLE 3
Tensile properties of crosslinked SPI/PEO electrospun
scaffolds at hydrated condition, n = 3

| | Young's modulus | Tensile strength | Strain at |
|---|---|---|---|
| Electrospun | (kPa) | (MPa) | break (%) |
| 7% SPI/3% PEO | 110 ± 6 | 0.06 ± 0.01 | 60 ± 3 |
| 12% SPI/3% PEO | 171 ± 21 | 0.17 ± 0.006 | 70 ± 12 |

Human Mesenchymal Stem Cell Culture.

Cell culture studies using hMSCs were carried out for 7% SPI/3% PEO and 12% SPI/3% PEO electrospun scaffolds to investigate cell behavior on electrospun SPI scaffolds. Fluorescence micrographs of live/dead stained electrospun scaffolds were obtained on 1 and 7 days of culture to assess cell viability (data not shown). Results indicated that the electrospun SPI/PEO scaffolds were able to support hMSC adhesion and viability. The cells were spread on the surface of the nanofiber mat exhibiting their normal phenotypic shape. Higher live cell densities were observed on day 7 for both types of electrospun scaffolds.

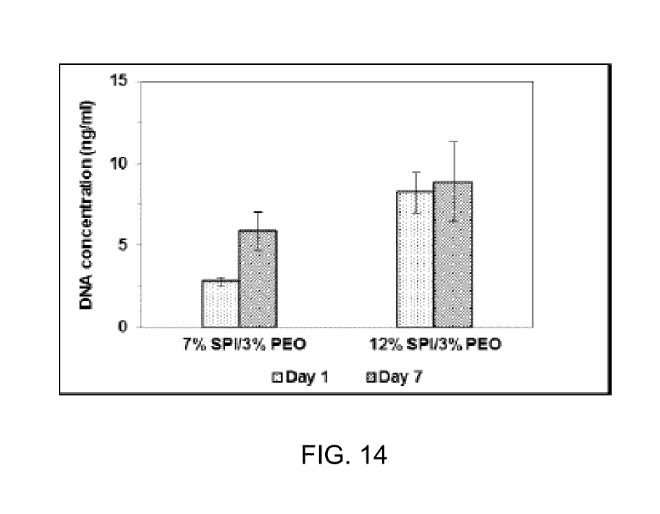

DNA was extracted from 7% SPI/3% PEO and 12% SPI/3% PEO electrospun scaffolds at each time point and quantified (FIG. 14) using Picogreen assay (n=4). Better cell attachment on day 1 was observed for 12% SPI/3% PEO electrospun scaffolds compared to 7% SPI/3% PEO scaffolds. This again may be due the presence of higher SPI content in 12% SPI/3% PEO potentially providing more protein-cell binding sites. Although a greater amount of cells were present on day 1 for the 12% SPI condition, a greater amount of cell proliferation was observed on the 7% SPI scaffolds resulting in a more than 2-fold increase in DNA amount for the 7% SPI scaffolds at day 7. The difference in proliferation rates may be due to the varying mechanical properties between the 12% SPI and 7% SPI. The higher protein content of the 12% SPI sample, which increased the mechanical properties of the scaffolds, may not have presented the ideal environment for hMSC proliferation.

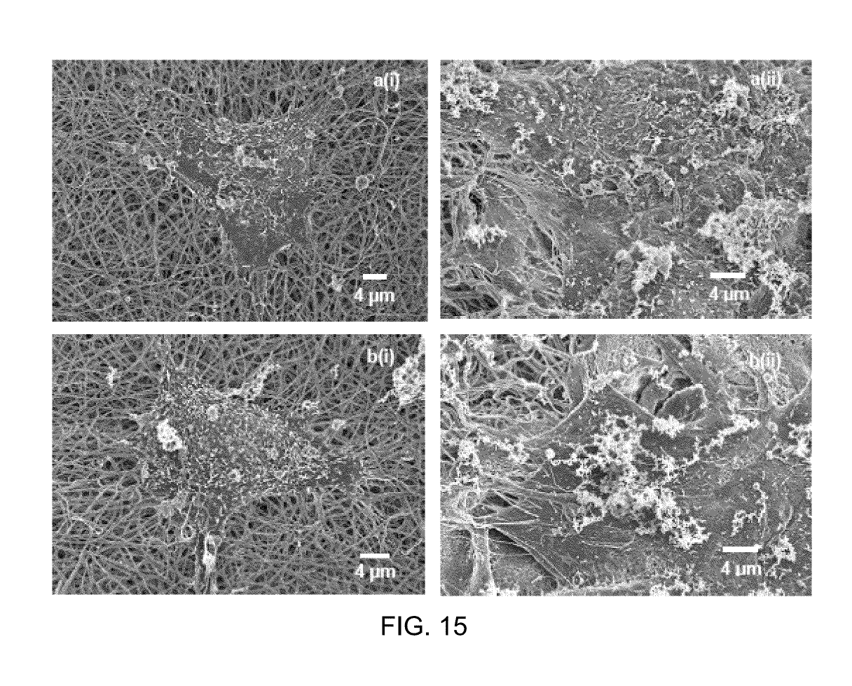

SEM images show hMSC attachment and spreading on SPI electrospun scaffolds (FIG. 15A-B). Clear interactions of cytoplasmic extensions of the hMSCs can be seen with the SPI fibers at both time points. Cells appeared to be completely integrated into the structure of the scaffold on day 7 with signs of matrix deposition on the surfaces surrounding the cells. These results illustrate the ability of SPI electrospun fibers to promote cell growth and biosynthesis. The collective results of this study demonstrate that novel SPI electrospun scaffolds can support cell viability and proliferation and have uses in regenerative medicine applications.

The word “illustrative” is used herein to mean serving as an example, instance, or illustration. Any aspect or design described herein as “illustrative” is not necessarily to be construed as preferred or advantageous over other aspects or designs. Further, for the purposes of this disclosure and unless otherwise specified, “a” or “an” means “one or more”. Still further, the use of “and” or “or” is intended to include “and/or” unless specifically indicated otherwise.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art, all language such as “up to,” “at least,” “greater than,” “less than,” and the like includes the number recited and refers to ranges which can be subsequently broken down into subranges as discussed above. Finally, as will be understood by one skilled in the art, a range includes each individual member.

The foregoing description of illustrative embodiments of the invention has been presented for purposes of illustration and of description. It is not intended to be exhaustive or to limit the invention to the precise form disclosed, and modifications and variations are possible in light of the above teachings or may be acquired from practice of the invention. The embodiments were chosen and described in order to explain the principles of the invention and as practical applications of the invention to enable one skilled in the art to utilize the invention in various embodiments and with various modifications as suited to the particular use contemplated. It is intended that the scope of the invention be defined by the claims appended hereto and their equivalents.

What is claimed is:

1. A porous soy protein-containing material comprising a plurality of partially denatured soy protein chains, wherein at least some of the soy protein chains are entangled and crosslinked to other soy protein chains.

2. The material of claim 1, having a total porosity of at least 80 volume percent.

3. The material of claim 1, wherein the material comprises substantially no other plant-based protein or plant-based polymer besides soy protein.

4. The material of claim 1, wherein substantially all the pores of the material are irregularly shaped pores in the form of elongated, tortuous channels.

5. The material of claim 1, wherein the soy protein chains are crosslinked with an enzymatic crosslinker at an amount of from about 0.1 units of enzymatic activity to about 1 units of enzymatic activity per gram of soy protein.

6. The material of claim 1, wherein the soy protein chains are crosslinked with the enzymatic crosslinker transglutaminase.

7. The material of claim 1, wherein the orientation of pores throughout the material is substantially random.

8. The material of claim 1, having a compressive modulus in the range of from about 30 Pa to about 70 Pa.

9. The material of claim 1, comprising lyophilized soy protein chains.

10. The material of claim 1, comprising lyophilized soy protein chains and wherein the soy protein chains are crosslinked with an enzymatic crosslinker at an amount of from about 0.1 units of enzymatic activity to about 1 units of enzymatic activity per gram of soy protein and further wherein the material comprises an oligosaccharide suitable for increasing cell metabolism.

11. The material of claim 1, comprising a mat of electrospun nanofibers, the nanofibers comprising the soy protein chains and one or more organic fiber-forming polymers.

12. The material of claim 11, wherein at least some of the nanofibers comprise soy protein chains crosslinked to soy protein chains of another nanofiber via one or more amide linkages.

13. The material of claim 11, wherein the organic fiber-forming polymer is poly(ethylene oxide) having a molecular weight in the range of from about 50 kDa to about 10 MDa.

14. The material of claim 11, wherein the nanofibers have an average fiber diameter in the range from about 90 nm to about 200 nm and the material has a porosity of at least 40 volume percent.

15. The material of claim 11, wherein the weight ratio of soy protein to organic fiber-forming polymer is in the range from about 4:1 to about 1:1.

16. The material of claim 11, wherein at least some of the nanofibers comprise soy protein chains crosslinked to soy protein chains of another nanofiber via one or more amide linkages, wherein the weight ratio of soy protein to organic fiber-forming polymer is in the range from about 4:1 to about 1:1, wherein the nanofibers have an average fiber diameter in the range from about 90 nm to about 200 nm, and wherein the material has a porosity of at least 40 volume percent and a Young's modulus in the range of from about 100 kPa to about 130 kPa.

17. The material of claim 1, wherein the material seeded with hMSC cells exhibits at least a two-fold increase in DNA content over a period of about seven days and the hMSC cells are integrated within the pores of the material.

18. A tissue growth scaffold comprising the material of claim 1 and tissue-forming cells, or cells that are precursors to tissue-forming cells, integrated within the pores of the porous soy protein-containing material.

19. A method of forming a porous soy protein-containing material comprising a plurality of partially denatured soy protein chains, wherein at least some of the soy protein chains are entangled and crosslinked to other soy protein chains, the method comprising:
reacting a crosslinker with partially denatured soy protein chains in a slurry comprising the soy protein chains, the crosslinker and a solvent under conditions that provide covalent linkages between at least some of the soy protein chains; and
lyophilizing the slurry to provide the porous soy protein-containing material.

20. A method of forming a porous soy protein-containing material comprising a plurality of partially denatured soy protein chains, wherein at least some of the soy protein chains are entangled and crosslinked to other soy protein chains:
electrospinning a first solution comprising partially denatured soy protein chains and organic fiber-forming molecules under conditions that provide a mat of nanofibers, wherein the nanofibers comprise the soy protein chains and the organic fiber-forming molecules; and
crosslinking the nanofibers in the mat by exposing them to a second solution comprising a crosslinker under conditions that provide covalent linkages between at least some of the soy protein chains of at least some nanofibers with soy protein chains of other nanofibers, thereby providing the porous soy protein-containing material.
